(19) United States
(12) Patent Application Publication
(10) Pub. No.: US 2023/0310294 A1
(43) Pub. Date: Oct. 5, 2023

(54) COMPOSITION FOR SIMULTANEOUS BLEACHING AND DYEING OF KERATIN FIBERS, COMPRISING A PARTICULAR HETEROCYCLIC SALT, AND PROCESS USING THIS COMPOSITION

(71) Applicant: L’Oreal, Paris (FR)

(72) Inventors: Stéphane BLAIS, AULNAY-SOUS-BOIS (FR); Stéphane SABELLE, AULNAY-SOUS-BOIS (FR)

(21) Appl. No.: 18/012,299

(22) PCT Filed: Jun. 18, 2021
(86) PCT No.: PCT/EP2021/066705

§ 371 Date: Dec. 22, 2022

(30) Foreign Application Priority Data
Jun. 23, 2020 (FR) 2006566

(51) Int. Cl. A61Q 5/08; A61Q 5/10; A61K 8/49; A61K 8/22

(52) U.S. Cl. CPC A61K 8/4946; A61K 8/22; A61Q 5/08; A61Q 5/10; A61K 2800/43; A61K 2800/87; A61K 2800/884

(57) ABSTRACT

The present invention relates to a composition for simultaneous bleaching and dyeing of keratin fibers, comprising one or more particular heterocyclic salts, and also to a process for simultaneous bleaching and dyeing of keratin fibers using this composition.

### FIELD OF THE INVENTION

The present invention relates to a composition for simultaneous bleaching and dyeing of keratin fibers, comprising one or more particular heterocyclic salts, and also to a process for simultaneous bleaching and dyeing of keratin fibers using this composition.

### BACKGROUND OF THE INVENTION

When a person wishes to radically change hair color, in particular when he or she wishes to obtain a lighter color than his or her original color, it is often necessary to carry out bleaching and then dyeing of the hair. There are several methods for doing this.

The first method consists in using lightening products based on aqueous ammonia and on hydrogen peroxide. These products may optionally contain dyes, thereby making it possible to simultaneously lighten and dye the hair. However, the lightening performance results of these products remain limited, more particularly for applications to natural and/or dyed dark-colored hair.

The second method consists in applying to the hair lightening compositions based on peroxygenated salts, such as persulfates, and on alkaline agents, to which hydrogen peroxide has been added at the time of use, in order to obtain greater lightening. These compositions may comprise direct dyes in order to simultaneously dye and bleach the hair. However, the range of direct dyes that can be used in these compositions remains restricted since only stable direct dyes can be used in such conditions in order to obtain good color build-up and intense and chromatic colors. In order to attempt to remedy this stability problem, it has been envisioned to perform the process in two steps: a first step during which the hair is bleached using the lightening composition, and then a second step during which the hair is dyed using a composition comprising direct dyes. However, a two-step process is not satisfactory since, in addition to creating numerous manipulations, it has the drawback of being relatively long.

There is therefore a real need to develop a composition for bleaching and dyeing keratin fibers, comprising direct dyes, the composition having both good lightening properties and good dyeing properties, particularly when it is applied to dark-colored hair, and making it possible in particular to obtain good color build-up and also intense and chromatic colors. Furthermore, such a composition may comprise a wide range of direct dyes making it possible to obtain the shade sought by the user. Finally, such a composition may be used in a single-step process for simultaneous bleaching and dyeing of keratin fibers.

The applicant has discovered, surprisingly, that all of these objectives can be achieved by the composition according to the present invention.

### SUMMARY OF THE INVENTION

According to a first aspect, a subject of the present invention is a composition comprising:
a) one or more chemical oxidizing agents chosen from hydrogen peroxide, hydrogen peroxide-generating agents other than peroxygenated salts, and mixtures thereof;
b) one or more direct dyes;
c) one or more heterocyclic salts of formula (A):

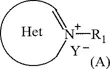

wherein:
   ▪ Het represents a cationic, aromatic, unsaturated heterocyclic group comprising:
      from 5 to 10 ring members, preferably 5 or 6 ring members; and
      in addition to the ammonium bearing R₁, one or two atoms chosen from nitrogen or oxygen atoms, preferably a nitrogen atom;
   said heterocyclic group being optionally substituted with one or more R′₁ or R₂ groups;
   ▪ R₁ and R′₁, which may be identical or different, represent a linear or branched, saturated or unsaturated C₁-C₁₂ hydrocarbon-based group optionally substituted notably with one or more groups chosen from hydroxyl, amino, (C₁-C₆)dialkylamino, (C₁-C₆)alkylamino, carboxyl, carboxylate, carbamide, (C₁-C₄)alkoxy, —SO₃H, sulfonate and phenyl;
   ▪ R₂ represents a hydroxyl radical, an amino radical, a linear or branched, saturated or unsaturated C₁-C₁₂ hydrocarbon-based group optionally substituted notably with one or more groups chosen from hydroxyl, amino, (C₁-C₆)dialkylamino, (C₁-C₆)alkylamino, carboxyl, carboxylate, carbamide, (C₁-C₄)alkoxy, —SO₃H, sulfonate and phenyl; and
   ▪ Y⁻ represents an anionic counterion;
   it being understood that:
   when one of the hydrocarbon-based groups of R₁, R′₁ or R₂ is substituted with a carboxylate or sulfonate group, then Y⁻ is absent to ensure the electrical neutrality of the salt of formula (A).

According to a second aspect, a subject of the present invention is a process for simultaneous bleaching and dyeing of keratin fibers, comprising the application to the keratin fibers of a composition as defined previously.

According to a third aspect, a subject of the present invention is a multi-compartment device or kit comprising:
▪ a first compartment containing a composition comprising a) one or more chemical oxidizing agents chosen from hydrogen peroxide, hydrogen peroxide-generating agents other than peroxygenated salts, and mixtures thereof; and
▪ a second compartment containing a composition comprising:
   b) one or more direct dyes; and
   c) one or more salts of formula (A) as defined previously; or
▪ a first compartment containing a composition comprising a) one or more chemical oxidizing agents chosen from hydrogen peroxide, hydrogen peroxide-generating agents other than peroxygenated salts, and mixtures thereof; and
▪ a second compartment containing a composition comprising b) one or more direct dyes; and
▪ a third compartment containing a composition comprising c) one or more salts of formula (A) as defined previously.

### DETAILED DESCRIPTION OF THE INVENTION

For the purposes of the present invention, and unless otherwise indicated:
▪ the term “keratin fibers” is intended to mean fibers of human or animal origin such as the hair, bodily hair, the eyelashes, the eyebrows, wool, angora, cashmere or fur. According to the present invention, the keratin fibers are preferably human keratin fibers, more preferentially the hair.
▪ the term “direct dyes” is intended to mean natural and/or synthetic dyes, including in the form of an extract or extracts, other than oxidation dyes. These are dyes that will spread superficially on the fiber. They may be ionic or nonionic, i.e. anionic, cationic, neutral or nonionic. Direct dyes may be of the same types of ionicity or else as mixtures.
▪ the direct dyes contain one or more chromophores, and these dyes are capable of absorbing light at a wavelength λ_{abs} ranging from 400 nm to 700 nm;
▪ the fluorescent direct dyes are dyes containing at least one fluorescent chromophore, and these dyes are capable of absorbing in the visible range at a wavelength λ_{abs} ranging from 400 nm to 800 nm and of re-emitting in the visible range at a wavelength λₑₘ longer than that absorbed ranging from 400 nm to 800 nm. The difference between the absorption and emission wavelengths, also known as the Stoke’s shift, is from 1 nm to 100 nm. Preferably, the fluorescent direct dyes are dyes that are capable of absorbing at a wavelength λ_{abs} ranging from 420 nm to 550 nm and of re-emitting in the visible range at a wavelength λₑₘ ranging from 470 nm to 600 nm;
▪ the term “chemical oxidizing agent” is intended to mean an oxidizing agent other than atmospheric oxygen.
▪ the term “alkyl group” is intended to mean a linear or branched, saturated hydrocarbon-based radical.
▪ the term “(Cₓ-C_{y})alkyl group” is intended to mean an alkyl group comprising from x to y carbon atoms.
▪ the term “hydroxy(Cₓ-C_{y})alkyl group” is intended to mean a (Cₓ-C_{y})alkyl group, at least one of the hydrogen atoms of which is replaced with a hydroxyl (—OH) group.
▪ the term “(hydroxy)(Cₓ-C_{y})alkyl group” is intended to mean a hydroxy(Cₓ-C_{y})alkyl group or a (Cₓ-C_{y})alkyl group.
▪ the term “alkoxy group” is intended to mean an alkyl group bonded to an oxygen atom.
▪ the term “(Cₓ-C_{y})alkoxy group” is intended to mean an alkoxy group comprising from x to y carbon atoms.
▪ the term “hydroxy(Cₓ-C_{y})alkoxy group” is intended to mean a (Cₓ-C_{y})alkoxy group, at least one of the hydrogen atoms of which is replaced with a hydroxyl (—OH) group.
▪ the expression “optionally substituted” applied to an alkyl or alkoxy group implies that the alkyl or alkoxy group may be substituted with one or more groups chosen from: i) hydroxyl, ii) (C₁-C₄)alkoxy, iii) acylamino, iv) amino optionally substituted with one or two identical or different (hydroxy)(C₁-C₄)alkyl groups, said alkyl groups possibly forming, with the nitrogen atom that bears them, a heterocycle comprising from 5 to 7 ring members, said heterocycle optionally comprising another nitrogen or non-nitrogen heteroatom; v) a quaternary ammonium group —N⁺R′R″R″′, M⁻ for which R′, R″ and R‴, which may be identical or different, represent a hydrogen atom, or a (C₁-C₄)alkyl group, or else R′, R″, R‴ form, with N⁺, a heteroaryl such as imidazolium optionally substituted with a (C₁-C₄)alkyl group, and M⁻ represents an anion, or a mixture of anions, intended to ensure electrical neutrality.
▪ the term “alkylene group” is intended to mean a linear or branched C₁-C₁₀, particularly C₁-C₆, more particularly C₁-C₂, acyclic hydrocarbon-based divalent chain, optionally substituted with one or more groups, which may be identical or different, chosen from i) hydroxyl, ii) (C₁-C₂)alkoxyl, iii) (poly)hydroxy(C₂-C₄)alkoxy(di)(C₁-C₂)(alkyl)amino, iv) (poly)hydroxy(di)(C₁-C₂)(alkyl)amino, v) R^{a}-z^{a}—C(Z^{b})—Z^{c}—, vi) R^{a′}—C(Z^{b})—Z^{a}— and vii) R^{a}—Z^{a}—S(O)ₜ—Z^{c}— with Z^{a}, Z^{b}, which may be identical or different, representing an oxygen or sulfur atom, or a group NR^{a}′, Z^{c} representing a bond, an oxygen or sulfur atom, or a group NR^{a}; R^{a} representing an alkali metal, a hydrogen atom or an alkyl group, or else is absent if another part of the molecule is cationic, and R^{a}′ representing a hydrogen atom or an alkyl group and t is equal to 1 or 2; preferably the “alkylene group” represents a —(CH₂)ₚ— group with p being an integer between 1 and 6, preferably between 1 and 4.
▪ the term “(hetero)aryl group” is intended to mean an aryl group or a heteroaryl group.
▪ the term “aryl group” is intended to mean a monocyclic or fused or non-fused polycyclic carbon-based group, comprising from 6 to 22 carbon atoms, at least one ring of which is aromatic; preferentially, the aryl group is a phenyl, biphenyl, naphthyl, indenyl, anthracenyl or tetrahydronaphthyl.
▪ the term “heteroaryl group” is intended to mean an optionally cationic, 5- to 22-membered monocyclic or fused or non-fused polycyclic group, comprising from 1 to 6 heteroatoms chosen from nitrogen, oxygen, sulfur and selenium atoms, and at least one ring of which is aromatic; preferentially, a heteroaryl group is chosen from acridinyl, benzimidazolyl, benzobistriazolyl, benzopyrazolyl, benzopyridazinyl, benzoquinolyl, benzothiazolyl, benzotriazolyl, benzoxazolyl, pyridyl, tetrazolyl, dihydrothiazolyl, imidazopyridyl, imidazolyl, indolyl, isoquinolyl, naphthoimidazolyl, naphthooxazolyl, naphthopyrazolyl, oxadiazolyl, oxazolyl, oxazolopyridyl, phenazinyl, phenoxazolyl, pyrazinyl, pyrazolyl, pyrilyl, pyrazoyltriazyl, pyridyl, pyridinoimidazolyl, pyrrolyl, quinolyl, tetrazolyl, thiadiazolyl, thiazolyl, thiazolopyridyl, thiazoylimidazolyl, thiopyrylyl, triazolyl, xanthylyl and its ammonium salt.
▪ a (hetero)aryl group or the aryl or heteroaryl part of a group may optionally be substituted with at least one group borne by a carbon atom, chosen from:
   a (C₁-C₁₀)alkyl, preferably (C₁-C₄)alkyl, group, optionally substituted with at least one group chosen from: hydroxyl, (C₁-C₂)alkoxy, hydroxy(C₂-C₄)alkoxy, acylamino, amino optionally substituted with one or two (C₁-C₄)alkyl groups, which may be identical or different, optionally bearing at least one hydroxyl group, or the two (C₁-C₄)alkyl groups possibly forming, with the nitrogen atom to which they are attached, a 5- to 7-membered, preferably 5- or 6-membered, heterocycle, said heterocycle being saturated or unsaturated and optionally substituted and optionally comprising another nitrogen or non-nitrogen heteroatom;
   a halogen atom;
   a hydroxyl group;
   a (C₁-C₂)alkoxy group;
   a (hydroxy)(C₂-C₄)alkoxy group;
   an amino group;
   a 5or 6-membered heterocycloalkyl group;
   an optionally cationic 5or 6-membered heteroaryl group, preferentially imidazolium, and optionally substituted with a (C₁-C₄)alkyl group, preferentially methyl;
   an amino group substituted with one or two identical or different (C₁-C₆)alkyl groups, optionally bearing at least:
      i) a hydroxyl group;
      ii) an amino group optionally substituted with one or two optionally substituted (C₁-C₃)alkyl groups, said alkyl groups possibly forming, with the nitrogen atom to which they are attached, a saturated or unsaturated, optionally substituted 5- to 7-membered heterocycle, optionally comprising at least one other nitrogen or non-nitrogen heteroatom;
      iii) a quaternary ammonium group —N⁺R′R″R″’, M⁻ for which R′, R″ and R‴, which may be identical or different, represent a hydrogen atom or a (C₁-C₄)alkyl group; and M⁻ represents an anionic counterion;
      iv) an optionally cationic 5- or 6-membered heteroaryl group, preferentially imidazolium, and optionally substituted with a (C₁-C₄)alkyl group, preferentially methyl;
      v) an acylamino (-N(R)—C(O)—R′) group wherein R represents a hydrogen atom or a (hydroxy)(C₁-C₄)alkyl group and R′ represents a (C₁-C₂)alkyl group;
   an acylamino (-N(R)—C(O)—R′) group wherein R represents a hydrogen atom or a (hydroxy)(C₁-C₄)alkyl group and R′ represents a (C₁-C₂)alkyl group;
   a carbamoyl ((R)₂N—C(O)—) group wherein R, which may be identical or different, represent a hydrogen atom or a (hydroxy)(C₁-C₄)alkyl group;
   an alkylsulfonylamino (R′—S(O)₂—N(R)-) group wherein R represents a hydrogen atom or a (hydroxy)(C₁-C₄)alkyl group and R′ represents a (C₁-C₄)alkyl group or a phenyl group;
   an aminosulfonyl ((R)₂N—S(O)₂—) group wherein R, which may be identical or different, represent a hydrogen atom or a (hydroxy)(C₁-C₄)alkyl group;
   a carboxylic group in acid or salified form (preferably salified with an alkali metal or a substituted or unsubstituted ammonium);
   a cyano group;
   a nitro or nitroso group;
   a polyhaloalkyl group, preferentially trifluoromethyl;
   an ester group.
▪ The term “cationic heteroaryl group” is intended to mean a heteroaryl group as defined previously, which comprises an endocyclic or exocyclic quaternized cationic group,
   when the cationic charge is endocyclic, it is included in the electron delocalization via the mesomeric effect; for example, it is a pyridinium, imidazolium or indolinium group: 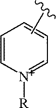 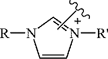 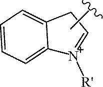
   with R and R′ being a heteroaryl substituent as defined previously and particularly a (hydroxy)(C₁-C₈)alkyl group such as methyl;
   when the charge is exocyclic, for example it is an ammonium substituent R⁺, such as trimethylammonium or phosphonium, which is exterior to the heteroaryl such as pyridyl, indolyl, imidazolyl or naphthalimidyl in question: 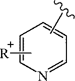 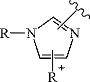 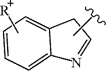 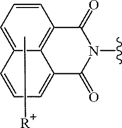 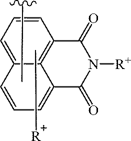
   with R a heteroaryl substituent as defined previously and R⁺ an ammonium RₐR_{b}R_{c}N⁺—, phosphonium RₐR_{b}R_{c}P⁺— or ammonium RₐR_{b}R_{c}N⁺-(C₁-C₆)alkylamino group with Rₐ, R_{b} and R_{c}, which may be identical or different, representing a hydrogen atom or a (C₁-C₈)alkyl group such as methyl.
▪ The term “cationic aryl group bearing an exocyclic charge” is intended to mean an aryl ring of which the quaternized cationic group is outside said ring; it is in particular an ammonium substituent R⁺ such as trimethylammonium or phosphonium, which is outside the aryl such as phenyl or naphthyl:

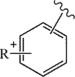

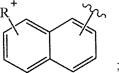

▪ the term “(hetero)cyclic group” is intended to mean a heterocyclic group or a carbocyclic group.
▪ the term “heterocyclic group” is intended to mean a 5- to 22-membered monocyclic or fused or non-fused polycyclic group that may contain one or two unsaturations but is nonaromatic, comprising from 1 to 6 heteroatoms chosen from nitrogen, oxygen, sulfur and selenium atoms.
▪ the term “heterocycloalkyl group” is intended to mean a saturated heterocyclic group such as morpholinyl, piperazinyl or pyperidinyl.
▪ the term “carbocyclic group” is intended to mean a 5- to 22-membered monocyclic or fused or non-fused polycyclic group that may contain one or two unsaturations but is nonaromatic, such as cyclobutyl, cyclopentyl or cyclohexyl.
▪ The carbocyclic or heterocyclic part of a nonaromatic group may optionally be substituted with at least one group chosen from:
   a hydroxyl group;
   a (C₁-C₄)alkoxy or hydroxy(C₂-C₄)alkoxy group;
   a (C₁-C₄)alkyl group;
   an alkylcarbonylamino (R—C(O)—N(R′)—) group wherein R′ is a hydrogen atom or a (hydroxy)(C₁-C₄)alkyl group and R is a (C₁-C₂)alkyl group or an amino group optionally substituted with one or two identical or different (hydroxy)(C₁-C₄)alkyl groups, said (hydroxy)(C₁-C₄)alkyl groups possibly forming, with the nitrogen atom to which they are attached, a saturated or unsaturated, optionally substituted 5- to 7-membered heterocycle optionally comprising at least one other nitrogen or non-nitrogen heteroatom;
   an alkylcarbonyloxy (R—C(O)—O—) group wherein R is a (C₁-C₄)alkyl group or an amino group optionally substituted with one or two identical or different (hydroxy)(C₁-C₄)alkyl groups, said (hydroxy)(C₁-C₄)alkyl groups possibly forming, with the nitrogen atom to which they are attached, a saturated or unsaturated, optionally substituted 5- to 7-membered heterocycle optionally comprising at least one other nitrogen or non-nitrogen heteroatom.
   an alkoxycarbonyl (R—G—C(O)—) group wherein R is a (C₁-C₄)alkyl group, G is an oxygen atom or an amino group optionally substituted with a (hydroxy)(C₁-C₄)alkyl group.
▪ a cyclic or heterocyclic group, or a nonaromatic part of an aryl or heteroaryl group, may also be substituted with at least one oxo group.
▪ a hydrocarbon-based chain is unsaturated when it includes one or more double bonds and/or one or more triple bonds.

Unless otherwise indicated, when compounds are mentioned in the present application, this also includes the optical isomers thereof, the geometrical isomers thereof, the tautomers thereof, the salts thereof or the solvates thereof, alone or as a mixture.

The expressions “at least one” and “one or more” are synonymous and may be used interchangeably.

### Composition

According to a first aspect, a subject of the present invention is a composition as defined previously.

The applicant has noted, surprisingly, that the addition of one or more heterocyclic salts of formula (A) to a composition comprising one or more oxidizing agents makes it possible to stabilize the direct dye(s) and to obtain a satisfactory level of lightening and also better color build-up and intense and chromatic colors.

### Chemical Oxidizing Agents A)

The composition according to the invention comprises a) one or more chemical oxidizing agents chosen from hydrogen peroxide, hydrogen peroxide-generating agents other than peroxygenated salts, and mixtures thereof.

The hydrogen peroxide-generating agents other than peroxygenated salts can be chosen from urea peroxide, polymeric complexes that can release hydrogen peroxide, oxidases, and mixtures thereof.

By way of example of polymeric complexes that can release hydrogen peroxide, mention may be made of polyvinylpyrrolidone/H₂O₂ in particular in the form of a powder, and the other polymeric complexes described in US 5 008 093, US 3 376 110 and US 5 183 901.

Oxidases can produce hydrogen peroxide in the presence of a suitable substrate, such as for example glucose in the case of glucose oxidase or uric acid with uricase.

Particularly preferably, the composition according to the invention comprises hydrogen peroxide as chemical oxidizing agent.

According to one particular embodiment, the hydrogen peroxide and/or the hydrogen peroxide-generating agent(s) other than peroxygenated salts can be added to the composition according to the invention just before it is applied to the keratin fibers. The intermediate composition(s) comprising the hydrogen peroxide and/or the hydrogen peroxide-generating agent(s) other than peroxygenated salts can be referred to as oxidizing compositions and can also contain various additional compounds or various adjuvants conventionally used in hair-lightening compositions.

The chemical oxidizing agent(s) may be present in the composition in a total content ranging from 0.1% to 40% by weight, preferably ranging from 1% to 20% by weight, more preferentially ranging from 2% to 15% by weight, relative to the total weight of the composition.

### Direct Dyes B)

The composition according to the invention comprises b) one or more direct dyes.

The direct dyes b) may be neutral, cationic or anionic direct dyes.

The direct dyes b) may be neutral, cationic or anionic direct dyes chosen from: acridines; acridones; anthranthrones; anthrapyrimidines; anthraquinones; azines; (poly)azos or azo, hydrazono or hydrazones, in particular arylhydrazones; azomethines; benzanthrones; benzimidazoles; benzimidazolones; benzindoles; benzoxazoles; benzopyrans; benzothiazoles; benzoquinones; bis-isoindolines; carboxanilides; coumarins; cyanines, such as (di)azacarbocyanines, (di)azahemicyanines, hemicyanines or tetraazacarbocyanines; (di)azines; bis-azines; (di)oxazines; (di)thiazines; (di)phenylamines; (di)phenylmethanes; (di)ketopyrrolopyrroles; flavonoids, such as flavanthrones and flavones; fluorindines; formazans; indamines; indanthrones; indigoids, thioindigoids and pseudoindigoids; indophenols; indoanilines; isoindolines; isoindolinones; isoviolanthrones; lactones; (poly)methines, such as dimethines of stilbene or styryl types; naphthalimides; naphthanilides; naphtholactams; naphthoquinones; nitro, notably nitro(hetero)aromatics; oxadiazoles; oxazines; perilones; perinones; perylenes; phenazines; phenoxazines; phenothiazines; phthalocyanine; polyenes/carotenoids; porphyrins; pyranthrones; pyrazolanthrones; pyrazolones; pyrimidinoanthrones; pyronines; quinacridones; quinolines; quinophthalones; squaranes; tetrazolines; thiazines; thiopyronines; triarylmethanes or xanthenes and natural direct dyes. Preferably, the direct dyes b) are chosen from anthraquinones, (poly)azos, azomethines and stilbenes, more preferentially from anthraquinones.

The direct dyes b) can be chosen in particular from neutral, cationic or anionic nitrobenzene direct dyes, neutral, cationic or anionic azo direct dyes, neutral, cationic or anionic tetraazapentamethine dyes, cationic or anionic quinone dyes and in particular neutral, cationic or anionic anthraquinone dyes, neutral, cationic or anionic azine direct dyes, neutral, cationic or anionic triarylmethane direct dyes, neutral, cationic or anionic azomethine direct dyes and natural direct dyes. Preferably, the direct dyes are chosen from neutral or anionic anthraquinone dyes and stilbenes.

By way of neutral, anionic or cationic direct dyes that can be used in the present invention, mention may be made of the following dyes: acridines; acridones; anthranthrones; anthrapyrimidines; anthraquinones; azines; (poly)azos, hydrazono or hydrazones, in particular arylhydrazones; azomethines; benzanthrones; benzimidazoles; benzimidazolones; benzindoles; benzoxazoles; benzopyrans; benzothiazoles; benzoquinones; bisazines; bis-isoindolines; carboxanilides; coumarins; cyanines, such as azacarbocyanines, diazacarbocyanines, diazahemicyanines, hemicyanines or tetraazacarbocyanines; diazines; diketopyrrolopyrroles; dioxazines; diphenylamines; diphenylmethanes; dithiazines; flavonoids, such as flavanthrones and flavones; fluorindines; formazans; indamines; indanthrones; indigoids and pseudoindigoids; indophenols; indoanilines; isoindolines; isoindolinones; isoviolanthrones; lactones; (poly)methines, such as dimethines of stilbene or styryl types; naphthalimides; naphthanilides; naphtholactams; naphthoquinones; nitro, notably nitro(hetero)aromatics; oxadiazoles; oxazines; perilones; perinones; perylenes; phenazines; phenoxazine; phenothiazines; phthalocyanine; polyenes/carotenoids; porphyrins; pyranthrones; pyrazolanthrones; pyrazolones; pyrimidinoanthrones; pyronines; quinacridones; quinolines; quinophthalones; squaranes; tetrazole; thiazines; thioindigo; thiopyronines; triarylmethanes or xanthenes.

### Neutral Direct Dyes

The direct dyes b) may be neutral direct dyes, preferably chosen from the hydrazono dyes of formulae (IIIa) and (III′a), the azo and styryl dyes (IVa), the diazo and distyryl dyes (IV′a) and (IV″a), the anthraquinone dyes (Va) and the azomethine dyes (VIa) and (VI′a) below, and mixtures thereof:

| | | | |
|---|---|---|---|
| Ar″—C(R^{a})═N—N(R^{b})—Ar (IIIa) | Ar″—N(R^{a})—N═C(R^{b})—Ar (III′a) | Ar—T═T′—Ar” (IVa) | Ar″—T═T′—Ar′—T′═T—Ar (IV′a) |
| Ar—T═T′—L—T′═T—Ar (IV″a) | 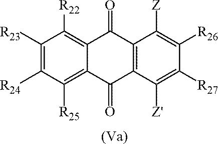 | | |
| 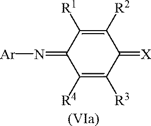 | | 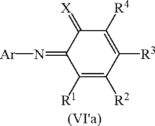 | |

formulae (IIIa), (III′a), (IVa), (IV′a), (IV″a), (Va), (VIa) and (VI′a) wherein:
▪ Ar represents an aryl group, such as phenyl or naphthyl, substituted with at least one electron-donating group such as i) optionally substituted (C₁-C₈)alkyl, ii) optionally substituted (C₁-C₈)alkoxy, iii) (di)(C₁-C₈)(alkyl)amino optionally substituted on the alkyl group(s) with a hydroxyl group, iv) aryl(C₁-C₈)alkylamino, v) optionally substituted N-(C₁-C₈)alkyl-N-aryl(C₁-C₈)alkylamino or Ar represents a julolidine group;
▪ Ar′ represents an optionally substituted divalent (hetero)arylene group, such as phenylene, particularly para-phenylene, or naphthalene, which is optionally substituted, preferably with one or more (C₁-C₈)alkyl, hydroxyl or (C₁-C₈)alkoxy group(s);
▪ Ar″ represents a (hetero)aryl group, which is optionally substituted, preferably with at least i) an electron-withdrawing group such as nitro, nitroso, —C(X)—X′—R′ or ii) a (di)(C₁-C₆)(alkyl)amino group, iii) hydroxyl, iv) (C₁-C₆)alkoxy; (hetero)aryl is particularly chosen from imidazolyl, triazolyl, indolyl or pyridyl or phenyl optionally substituted with at least one group chosen from nitro, nitroso and amino, preferably substituted in the position para to the phenyl group;
▪ X, X′ and X″, which may be identical or different, represent an oxygen or sulfur atom, or a group NR”, preferably oxygen;
▪ R¹, R², R³ and R⁴, which may be identical or different, represent a hydrogen or halogen atom, or a group chosen from hydroxyl, thiol, (C₁-C₄)alkyl, (C₁-C₄)alkoxy, (di)(C₁-C₄)(alkyl)amino, nitro and nitroso;
▪ R′ and R″ represent a (C₁-C₄)alkyl group;
▪ R^{a} and R^{b}, which may be identical or different, represent a hydrogen atom or a (C₁-C₈)alkyl group, which is optionally substituted, preferably with a hydroxyl group;
   or, as a variant, the substituent R^{a} with a substituent of Ar″ and/or R^{b} with a substituent of Ar and/or R^{a} with R^{b} form, together with the atoms that bear them, a (hetero)cycloalkyl; in particular, R^{a} and R^{b} represent a hydrogen atom or a (C₁-C₄)alkyl group, which is optionally substituted with a hydroxyl group;
▪ T and T′, which may be identical or different, represent a group C(R^{a}) or N, preferably N; and
▪ L represents a divalent group -ALK-, -C(X)-ALK-, -ALK-C(X)- or -C(X)-ALK-C(X′)- with ALK representing a linear or branched (C₁-C₆)alkylene group, such as methylene, and X and X′ as defined previously;
▪ R₂₂, R₂₃, R₂₄, R₂₅, R₂₆ and R₂₇, which may be identical or different, represent a hydrogen or halogen atom, or a group chosen from:
   (C₁-C₆)alkyl;
   hydroxyl, mercapto;
   (C₁-C₆)alkoxy, (C₁-C₆)alkylthio;
   aryloxy or arylthio;
   aryl(C₁-C₆)(alkyl)amino;
   (di)(C₁-C₆)(alkyl)amino;
   (di)(hydroxy(C₁-C₆)alkyl)amino;
▪ Z′ represents a hydrogen atom or a group NR₂₈R₂₉ with R₂₈ and R₂₉, which may be identical or different, representing a hydrogen atom or a group chosen from:
   (C₁-C₆)alkyl;
   polyhydroxy(C₁-C₆)alkyl such as hydroxyethyl;
   aryl optionally substituted with one or more group(s), particularly i) (C₁-C₆)alkyl; iii) R°—C(X)—X′—, R°—X′—C(X)—, R°—X′—C(X)—X″— with R° representing a (C₁-C₆)alkyl group and X, X′ and X″ as defined previously; iv) a sulfonate;
   cycloalkyl; notably cyclohexyl;
▪ Z represents a group chosen from hydroxyl and NR′₂₈R′₂₉ with R′₂₈ and R′₂₉, which may be identical or different, representing the same atoms or groups as R₂₈ and R₂₉ as defined previously.

The direct dyes b) of formula (IV″a) are preferably of formula (IV‴a)

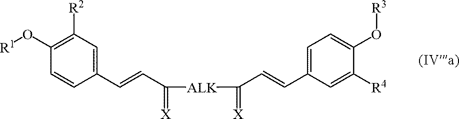

Formula (IV‴a) wherein:
▪ R¹ and R³, which may be identical or different, preferably identical, represent a hydrogen atom, a (C₁-C₄)alkyl group such as methyl or a sugar such as glucosyl, preferably a hydrogen atom;
▪ R² and R⁴, which may be identical or different, preferably identical, represent a hydrogen atom, a (C₁-C₄)alkyl or (C₁-C₄)alkoxy group or an —O—sugar group such as —O—glucosyl, preferably (C₁-C₄)alkoxy; such as methoxy;
▪ X, which may be identical or different, preferably identical, represents an oxygen or sulfur atom or N-R with R representing a hydrogen atom or a group, preferably an oxygen atom;
▪ ALK represents a (C₁-C₄)alkylene group such as methylene or ethylene, preferably methylene.

The direct dyes of formula (IV″a) may be derived from curcumin, demethoxycurcumin and bis-demethoxycurcumin.

Preferably, the direct dyes b) are chosen from the direct dyes of formulae (IV″a) and (IV‴a) and mixtures thereof as defined previously.

According to one particularly preferred embodiment, the direct dyes b) are neutral direct dyes chosen from the following compounds (A) to (G) and mixtures thereof:

| | | |
|---|---|---|
| 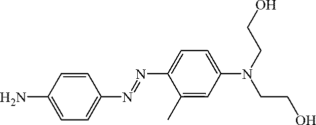 [SMILES: Cc1cc(N(CCO)CCO)ccc1/N=N/c1ccc(N)cc1] | 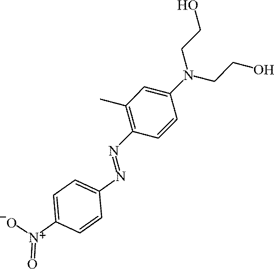 [SMILES: Cc1cc(N(CCO)CCO)ccc1/N=N/c1ccc([N+](=O)[O-])cc1] | 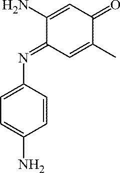 [SMILES: CC1=C/C(=N\c2ccc(N)cc2)C(N)=CC1=O] |
| (A) | (B) | (C) |
| 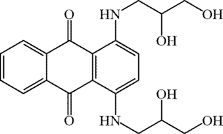 [SMILES: O=C1c2ccccc2C(=O)c2c(NCC(O)CO)ccc(NCC(O)CO)c21] | 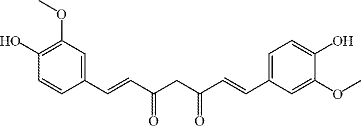 [SMILES: COc1cc(/C=C/C(=O)CC(=O)/C=C/c2ccc(O)c(OC)c2)ccc1O] | |
| (D) | (E) | |
| 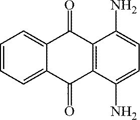 [SMILES: Nc1ccc(N)c2c1C(=O)c1ccccc1C2=O] | 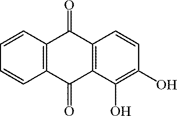 [SMILES: O=C1c2ccccc2C(=O)c2c1ccc(O)c2O] | |
| (F) | (G) | |

preferably from the compounds (E), (F) and (G) and mixtures thereof, more preferentially from the compounds (E) and (G) and mixtures thereof.

### Cationic Direct Dyes

The direct dyes b) can be chosen from direct dyes which are cationic or commonly referred to as “basic dyes” for their affinity with acidic substances comprising in particular in their structure at least one endocyclic or exocyclic, cationic or cationizable group.

As cationic azo dyes that can be used in the present invention, mention may be made particularly of the cationic dyes described in Kirk-Othmer’s Encyclopedia of Chemical Technology, “Dyes, Azo”, J. Wiley & Sons, updated on Apr. 19, 2010.

Mention may also be made of the cationic azo dyes described in patent applications WO 95/15144, WO 95/01772 and EP 714954.

Mention may also be made of the cationic azo dyes described in the Color Index International, 3rd edition, in particular of the following compounds: Basic Red 22; Basic Red 76; Basic Yellow 57; Basic Brown 16; Basic Brown 17.

Among the cationic quinone dyes, those mentioned in the Color Index International, 3rd edition, are suitable and, among these, mention may be made, inter alia, of the following dyes: Basic Blue 22; Basic Blue 99.

Among the azine dyes that are suitable, mention may be made of those listed in the Color Index International, 3rd edition, and for example of the following dyes: Basic Blue 17, Basic Red 2.

Among the cationic triarylmethane dyes that can be used according to the invention, mention may be made, in addition to those listed in the Color Index International, 3rd edition, of the following dyes: Basic Green 1, Basic Violet 3, Basic Violet 14, Basic Blue 7, Basic Blue 26.

Mention may also be made of the direct dyes described in documents US 5 888 252, EP 1 133 975, WO 03/029 359, EP 860 636, WO 95/01772, WO 95/15144 and EP 714 954. Mention may also be made of those listed in the encyclopedia “The Chemistry of Synthetic Dyes” by K. Venkataraman, 1952, Academic Press, vol. 1 to 7, in “Kirk-Othmer’s Encyclopedia of Chemical Technology”, in the chapter “Dyes and Dye Intermediates”, 1993, Wiley and Sons, and in various chapters of “Ullmann’s Encyclopedia of Industrial Chemistry”, 7th edition, Wiley and Sons.

Preferably, the cationic direct dyes are chosen from those resulting from dyes of azo and hydrazono type.

The cationic direct dyes may be cationic azo dyes, as described in EP 850 636,FR 2 788 433,, EP 920 856,WO 99/48465,FR 2 757 385,EP 850 637,EP 918 053,WO 97/44004,FR 2 570 946,FR 2 285 851,DE 2 538 363,FR 2 189 006,FR 1 560 664,FR 1 540 423,FR 1 567 219,FR 1 516 943,FR 1 221 122,DE 4 220 388,DE 4 137 005, WO 01/66646, US 5 708 151, WO 95/01772, WO 515 144, GB 1 195 386, US 3 524 842, US 5 879 413, EP 1 062 940, EP 1 133 976, GB 738 585, DE 2 527 638FR 2 275 462, GB 1974-27645, Acta Histochem. (1978), 61(1), 48-52; Tsitologiya (1968), 10(3), 403-5; Zh. Obshch. Khim., (1970), 40(1), 195-202; Ann. Chim. (Rome) (1975), 65(5-6), 305-14; Journal of the Chinese Chemical Society (Taipei) (1998), 45(1), 209-211; Rev. Roum. Chim. (1988), 33(4), 377-83; Text. Res. J. (1984), 54(2), 105-7; Chim. Ind. (Milan) (1974), 56(9), 600-3; Khim. Tekhnol. (1979), 22(5), 548-53; Ger. Monatsh. Chem. (1975), 106(3), 643-8; MRL Bull. Res. Dev. (1992), 6(2), 21-7; Lihua Jianyan, Huaxue Fence (1993), 29(4), 233-4; Dyes Pigm. (1992), 19(1), 69-79; Dyes Pigm. (1989), 11(3), 163-72.

Preferably, the cationic direct dyes comprise a quaternary ammonium group; more preferentially, the cationic charge is endocyclic. These cationic groups are, for example, a cationic group:
bearing a (di/tri)(C₁-C₈)alkylammonium exocyclic charge, or
bearing an endocyclic charge, such as comprising a cationic heteroaryl group chosen from: acridinium, benzimidazolium, benzobistriazolium, benzopyrazolium, benzopyridazinium, benzoquinolium, benzothiazolium, benzotriazolium, benzoxazolium, bi-pyridinium, bis-tetrazolium, dihydrothiazolium, imidazopyridinium, imidazolium, indolium, isoquinolium, naphthoimidazolium, naphthoxazolium, naphthopyrazolium, oxadiazolium, oxazolium, oxazolopyridinium, oxonium, phenazinium, phenooxazolium, pyrazinium, pyrazolium, pyrazoyltriazolium, pyridinium, pyridinoimidazolium, pyrrolium, pyrylium, quinolium, tetrazolium, thiadiazolium, thiazolium, thiazolopyridinium, thiazoylimidazolium, thiopyrylium, triazolium or xanthylium.

Mention may be made of the hydrazono cationic direct dyes of formulae (IIb) and (IIIb) and the azo dyes of formulae (IVb) and (Vb) below:

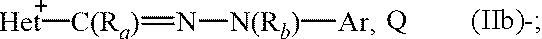

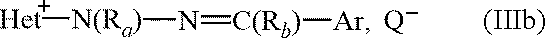

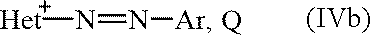

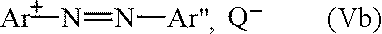

formulae (IIb) to (Vb) wherein:
▪ Het⁺ represents a cationic heteroaryl group, preferentially bearing an endocyclic cationic charge, such as imidazolium, indolium or pyridinium, which is optionally substituted, preferentially with at least one (C₁-C₈)alkyl group such as methyl;
▪ Ar⁺ represents an aryl group, such as phenyl or naphthyl, bearing an exocyclic cationic charge, preferentially ammonium, particularly tri(C₁-C₈)alkylammonium, such as trimethylammonium;
▪ Ar represents an aryl group, in particular phenyl, which is optionally substituted, preferentially with one or more electron-donating groups such as i) optionally substituted (C₁-C₈)alkyl, ii) optionally substituted (C₁-C₈)alkoxy, iii) (di)(C₁-C₈)(alkyl)amino optionally substituted on the alkyl group(s) with a hydroxyl group, iv) aryl(C₁-C₈)alkylamino, v) optionally substituted N-(C₁-C₈)alkyl-N-aryl(C₁-C₈)alkylamino or alternatively Ar represents a julolidine group;
▪ Ar″ represents an optionally substituted (hetero)aryl group, such as phenyl or pyrazolyl, which are optionally substituted, preferentially with one or more (C₁-C₈)alkyl, hydroxyl, (di)(C₁-C₈)(alkyl)amino, (C₁-C₈)alkoxy or phenyl groups;
▪ Rₐ and R_{b}, which may be identical or different, represent a hydrogen atom or a (C₁-C₈)alkyl group, which is optionally substituted, preferentially with a hydroxyl group;
▪ or else the substituent Rₐ with a substituent of Het⁺ and/or R_{b} with a substituent of Ar and/or Rₐ with R_{b} form, together with the atoms that bear them, a (hetero)cycloalkyl; in particular, Rₐ and R_{b} represent a hydrogen atom or a (C₁-C₄)alkyl group optionally substituted with a hydroxyl group;
▪ Q⁻ represents an anionic counterion such as a halide, an alkyl sulfate or an alkyl sulfonate.

In particular, mention may be made of the azo and hydrazono direct dyes bearing an endocyclic cationic charge of formulae (IIb) to (Vb) as defined previously. More particularly, mention may be made of the cationic direct dyes of formulae (IIb) to (Vb) bearing an endocyclic cationic charge described in patent applications WO 95/15144, WO 95/01772 and EP 714 954.

Preferably, mention may be made of the following direct dyes:

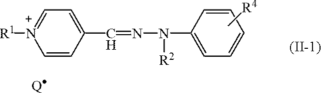

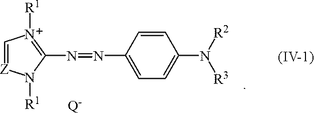

formulae (II-1) and (IV-1) wherein:
▪ R¹ represents a (C₁-C₄)alkyl group such as methyl;
▪ R² and R³, which may be identical or different, represent a hydrogen atom or a (C₁-C₄)alkyl group, such as methyl;
▪ R⁴ represents a hydrogen atom or an electron-donating group such as optionally substituted (C₁-C₈)alkyl, optionally substituted (C₁-C₈)alkoxy, or (di)(C₁-C₈)(alkyl)amino optionally substituted on the alkyl group(s) with a hydroxyl group; particularly, R⁴ is a hydrogen atom,
▪ Z represents a CH group or a nitrogen atom, preferentially CH,
▪ Q⁻ is an anionic counterion as defined previously, in particular a halide, such as chloride, or an alkyl sulfate, such as methyl sulfate or mesityl.

In particular, the dyes of formulae (II-1) and (IV-1) are chosen from Basic Red 51, Basic Yellow 87 and Basic Orange 31 or derivatives thereof:

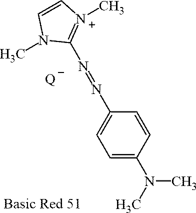

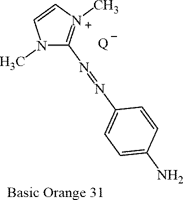

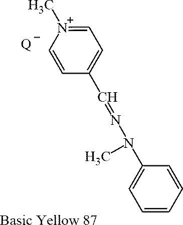

with Q′ being an anionic counterion as defined previously, in particular a halide, such as chloride, or an alkyl sulfate, such as methyl sulfate or mesityl.

### Fluorescent Dyes

The direct dyes b) can be chosen from fluorescent direct dyes.

By way of example of fluorescent dyes that may be used in the present invention, mention may be made of neutral, anionic or cationic dyes chosen from the following dyes: acridines, acridones, benzanthrones, benzimidazoles, benzimidazolones, benzindoles, benzoxazoles, benzopyrans, benzothiazoles, coumarins, difluoro{2-[(2H-pyrrol-2-ylidene-kN)methyl]-1H-pyrrolato-kN}borons (BODIPY®), diketopyrrolopyrroles, fluorindines, (poly)methines (in particular cyanines and styryls/hemicyanines), naphthalimides, naphthanilides, naphthylamine (such as dansyls), oxadiazoles, oxazines, perilones, perinones, perylenes, polyenes/carotenoids, squaranes, stilbenes, xanthenes.

Mention may also be made of the fluorescent dyes described in documents EP 1 133 975, WO 03/029 359, EP 860 636, WO 95/01772, WO 95/15144 and EP 714 954 and those listed in the encyclopedia “The Chemistry of Synthetic Dyes” by K. Venkataraman, 1952, Academic Press, vol. 1 to 7, in “Kirk-Othmer’s Encyclopedia of Chemical Technology”, in the chapter “Dyes and Dye Intermediates”, 1993, Wiley and Sons, and in various chapters of “Ullmann’s Encyclopedia of Industrial Chemistry”, 7th edition, Wiley and Sons, and in “The Handbook - A Guide to Fluorescent Probes and Labeling Technologies”, 10th Ed., Molecular Probes/Invitrogen - Oregon 2005, circulated on the Internet or in the preceding printed editions.

According to a preferred variant, the fluorescent dye(s) are cationic polymethines and comprise at least one quaternary ammonium group, such as those of formula (Vb) below: W⁺-[C(R_{c})=C(R_{d})_{m′}-Ar, Q⁻ formula (Vb) wherein:
▪ W⁺ represents a cationic heterocyclic or heteroaryl group, particularly comprising a quaternary ammonium optionally substituted with one or more (C₁-C₈)alkyl groups, optionally substituted notably with one or more hydroxyl groups;
▪ Ar representing an aryl group such as phenyl or naphthyl, optionally substituted preferentially with i) one or more halogen atoms, such as chlorine or fluorine; ii) one or more (C₁-C₈)alkyl, preferably (C₁-C₄)alkyl, groups such as methyl; iii) one or more hydroxyl groups; iv) one or more (C₁-C₈)alkoxy groups such as methoxy; v) one or more hydroxy(C₁-C₈)alkyl groups such as hydroxyethyl, vi) one or more amino or (di)(C₁-C₈)alkylamino groups, preferably with the C₁-C₄ alkyl part optionally substituted with one or more hydroxyl groups, such as (di)hydroxyethylamino, vii) one or more acylamino groups; viii) one or more heterocycloalkyl groups such as piperazinyl, piperidyl or 5- or 6-membered heteroaryl such as pyrrolidinyl, pyridyl and imidazolinyl;
▪ m′ represents an integer ranging from 1 to 4, preferably m′ is equal to 1 or 2; more preferentially m′ = 1;
▪ R_{c} and R_{d}, which may be identical or different, represent a hydrogen atom or an optionally substituted (C₁-C₈)alkyl group, preferentially an optionally substituted (C₁-C₄)alkyl group, or else R_{c} contiguous with W⁺ and/or R_{d} contiguous with Ar form, with the atoms that bear them, a (hetero)cycloalkyl; particularly, R_{c} is contiguous with W⁺ and they form a (hetero)cycloalkyl such as cyclohexyl;
▪ Q⁻ is an anionic counterion as defined previously.

### Anionic Dyes

The direct dyes b) can be chosen from anionic direct dyes or dyes commonly referred to as “acidic” direct dyes on account of their affinity with alkaline substances.

The term “anionic direct dyes” is intended to mean any direct dye comprising in its structure at least one CO₂R or SO₃R substituent with R denoting a hydrogen atom or a cation originating from a metal or from an amine, or an ammonium ion. The anionic dyes can be chosen from acid nitro direct dyes, acid azo dyes, acid azine dyes, acid triarylmethane dyes, acid indoamine dyes, acid anthraquinone dyes, indigoids and acid natural dyes.

Preferably, the anionic direct dyes are acidic anthraquinones.

The direct dyes b) can be anionic direct dyes preferably chosen from the dyes of formulae (III), (III′), (IV), (IV′), (V), (V′), (VI), (VI′), (VII), (VIII), (IX) and (X) below, and mixtures thereof:
a) the diaryl anionic azo dyes of formula (III) or (III′):

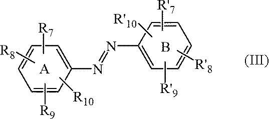

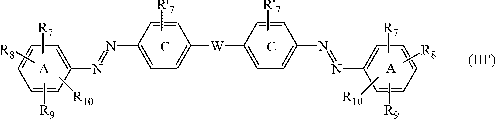

formulae (III) and (III′) wherein:
   ▪ R₇, R₈, R₉, R₁₀, R′₇, R′₈, R′₉ and R′₁₀, which may be identical or different, represent a hydrogen atom or a group chosen from:
      (C₁-C₆)alkyl;
      (C₁-C₆)alkoxy, (C₁-C₆)alkylthio;
      hydroxyl, mercapto;
      nitro, nitroso;
      R°-C(X)-X′-, R°-X′-C(X)-, R°-X′-C(X)-X″with R° representing a hydrogen atom or a (C₁-C₆)alkyl or aryl group such as phenyl; X, X′ and X″, which may be identical or different, representing an oxygen or sulfur atom, or NR with R representing a hydrogen atom or a (C₁-C₆)alkyl group;
      — (O)₂S(O⁻)—, M⁺ with M⁺ representing a hydrogen atom or a cationic counterion;
      — (O)CO⁻—, M⁺ with M⁺ as defined previously;
      — R″—S(O)₂—, with R″ representing a hydrogen atom, an alkyl group, or an aryl, (di)(C₁-C₆)(alkyl)amino, or aryl(C₁-C₆)(alkyl)amino group; preferentially a phenylamino or phenyl group;
      — R″′—S(O)₂—X′— with R‴ representing a (C₁-C₆)alkyl group or an aryl group which is optionally substituted, X′ as defined previously;
      (di)(C₁-C₆)(alkyl)amino;
      aryl(C₁-C₆)(alkyl)amino optionally substituted with one or more group(s) chosen from i) nitro; ii) nitroso; iii) (O)₂S(O⁻)—, M⁺ and iv) (C₁-C₆)alkoxy with M⁺ as defined previously;
      optionally substituted heteroaryl; preferentially a benzothiazolyl group;
      cycloalkyl; in particular cyclohexyl;
      — Ar—N═N— with Ar representing an optionally substituted aryl group; preferentially a phenyl optionally substituted with one or more alkyl, (O)₂S(O⁻)—, M⁺ or phenylamino group(s);
      or else two contiguous groups R₇ with R₈ or R₈ with R₉ or R₉ with R₁₀ together form a fused benzo group A′; and R′₇ with R′₈ or R′₈ with R′₉ or R′₉ with R′₁₀ together form a fused benzo group B′; with A′ and B′ optionally substituted with one or more group(s) chosen from i) nitro; ii) nitroso; iii) (O)₂S(O⁻)—, M⁺; iv) hydroxyl; v) mercapto; vi) (di)(alkyl)amino; vii) R°—C(X)—X′—; viii) R°-X′-C(X)-; ix) R°-X′-C(X)-X″-; x) Ar—N═N— and xi) optionally substituted aryl(C₁-C₆)(alkyl)amino; with M⁺, R°, X, X′, X″ and Ar as defined previously;
   ▪ W represents a sigma σ bond, an oxygen or sulfur atom, or a divalent group i) —NR—with R as defined previously, or ii) methylene —C(Rₐ)(R_{b})— with Rₐ and R_{b}, which may be identical or different, representing a hydrogen atom or an aryl group, or alternatively Rₐ and R_{b} form, together with the carbon atom that bears them, a spiro cycloalkyl; preferentially, W represents a sulfur atom or Rₐ and R_{b} together form a cyclohexyl; it being understood that the formulae (III) and (III′) comprise, on one of the rings A, A′, B, B′ or C:
      at least one group (O)₂S(O⁻)—, M′⁺ with M′⁺ representing a cationic counterion; or
      at least one group (O)CO⁻—, M′⁺ with M′⁺ representing a cationic counterion; preferably at least one sodium sulfonate group.

As examples of dyes of formula (III), mention may be made of: Acid Red 1, Acid Red 4, Acid Red 13, Acid Red 14, Acid Red 18, Acid Red 27, Acid Red 28, Acid Red 32, Acid Red 33, Acid Red 35, Acid Red 37, Acid Red 40, Acid Red 41, Acid Red 42, Acid Red 44, Pigment Red 57, Acid Red 68, Acid Red 73, Acid Red 135, Acid Red 138, Acid Red 184, Food Red 1, Food Red 13, Acid Orange 6, Acid Orange 7, Acid Orange 10, Acid Orange 19, Acid Orange 20, Acid Orange 24, Yellow 6, Acid Yellow 9, Acid Yellow 36, Acid Yellow 199, Food Yellow 3, Acid Violet 7, Acid Violet 14, Acid Blue 113, Acid Blue 117, Acid Black 1, Acid Brown 4, Acid Brown 20, Acid Black 26, Acid Black 52, Food Black 1, Food Black 2, Food Yellow 3 or Sunset Yellow;
and as examples of dyes of formula (III′), mention may be made of: Acid Red 111, Acid Red 134, Acid Yellow 38;
b) the pyrazolone anionic azo dyes of formula (IV) or (IV′):

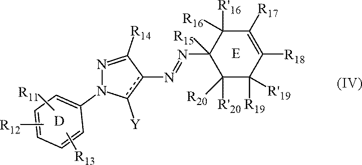

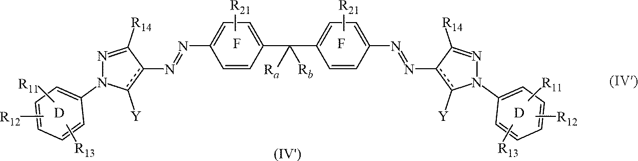

formulae (IV) and (IV′) wherein:
   ▪ R₁₁, R₁₂ and R₁₃, which may be identical or different, represent a hydrogen or halogen atom, or a (C₁-C₆)alkyl or (O)₂S(O⁻), M⁺ group with M⁺ as defined previously;
   ▪ R₁₄ represents a hydrogen atom, a (C₁-C₆)alkyl group or a —C(O)O⁻, M⁺ group with M⁺ as defined previously;
   ▪ R₁₅ represents a hydrogen atom;
   ▪ R₁₆ represents an oxo group, in which case R′₁₆ is absent, or else R₁₅ with R₁₆ together form a double bond;
   ▪ R₁₇ and R₁₈, which may be identical or different, represent a hydrogen atom, or a group chosen from:
      — (O)₂S(O⁻)—, M⁺ with M⁺ as defined previously;
      — Ar—O—S(O)₂— with Ar representing an optionally substituted aryl group; preferentially a phenyl optionally substituted with one or more alkyl group(s);
   ▪ R₁₉ and R₂₀ together form either a double bond, or a benzo group D′, which is optionally substituted;
   ▪ R′₁₆, R′₁₉ and R′₂₀, which may be identical or different, represent a hydrogen atom or a (C₁-C₆)alkyl group, or a hydroxyl group;
   ▪ R₂₁ represents a hydrogen atom or a (C₁-C₆)alkyl or (C₁-C₆)alkoxy group;
   ▪ Rₐ and Rₐ, which may be identical or different, are as defined previously; preferentially, Rₐ represents a hydrogen atom and R_{b} represents an aryl group such as phenyl;
   ▪ Y represents either a hydroxyl group or an oxo group;
   ▪ 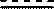
   represents a single bond when Y is an oxo group; and represents a double bond when Y represents a hydroxyl group;
      it being understood that the formulae (IV) and (IV′) comprise, on one of the rings D or E:
         at least one group (O)₂S(O⁻)—, M′⁺ with M′⁺ representing a cationic counterion; or
         at least one group (O)CO⁻—, M′⁺ with M′⁺ representing a cationic counterion; preferably at least one sodium sulfonate group.

As examples of dyes of formula (IV), mention may be made of: Acid Red 195, Acid Yellow 23, Acid Yellow 27, Acid Yellow 76, and as examples of dyes of formula (IV′), mention may be made of: Acid Yellow 17;
c) the anthraquinone dyes of formula (V) or (V′):

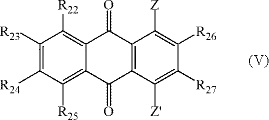

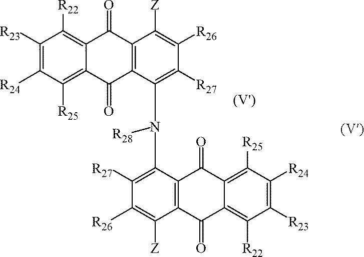

formulae (V) and (V′) wherein:
   ▪ R₂₂, R₂₃, R₂₄, R₂₅, R₂₆ and R₂₇, which may be identical or different, represent a hydrogen or halogen atom, or a group chosen from:
      (C₁-C₆)alkyl;
      hydroxyl, mercapto;
      (C₁-C₆)alkoxy, (C₁-C₆)alkylthio;
      optionally substituted aryloxy or arylthio, preferentially substituted with one or more group(s) chosen from alkyl and (O)₂S(O⁻)—, M⁺ with M⁺ as defined previously;
      aryl(C₁-C₆)(alkyl)amino optionally substituted with one or more group(s) chosen from alkyl and (O)₂S(O⁻)—, M⁺ with M⁺ as defined previously;
      (di)(C₁-C₆)(alkyl)amino;
      (di)(hydroxy(C₁-C₆)alkyl)amino;
      — (O)₂S(O⁻)—, M⁺ with M⁺ as defined previously;
   ▪ Z′ represents a hydrogen atom or a group NR₂₈R₂₉ with R₂₈ and R₂₉, which may be identical or different, representing a hydrogen atom or a group chosen from:
      (C₁-C₆)alkyl;
      polyhydroxy(C₁-C₆)alkyl such as hydroxyethyl;
      aryl optionally substituted with one or more group(s), particularly i) (C₁-C₆)alkyl such as methyl, n-dodecyl, n-butyl; ii) (O)₂S(O⁻)—, M⁺ with M⁺ as defined previously; iii) R°-C(X)-X′-, R°-X′-C(X)-, R°-X′-C(X)-X″- with R°, X, X′ and X″ as defined previously; preferentially R° represents a (C₁-C₆)alkyl group;
      cycloalkyl, notably cyclohexyl;
   ▪ Z represents a group chosen from hydroxyl and NR′₂₈R′₂₉ with R′₂₈ and R′₂₉, which may be identical or different, representing the same atoms or groups as R₂₈ and R₂₉ as defined previously;
      it being understood that the formulae (V) and (V′) comprise:
         at least one group (O)₂S(O⁻)—, M′⁺ with M′⁺ representing a cationic counterion; or
         at least one group (O)CO⁻—, M′⁺ with M′⁺ representing a cationic counterion; preferably at least one sodium sulfonate group.

As examples of dyes of formula (V), mention may be made of: Acid Blue 25, Acid Blue 43, Acid Blue 62, Acid Blue 78, Acid Blue 129, Acid Blue 138, Acid Blue 140, Acid Blue 251, Acid Green 25, Acid Green 41, Acid Violet 42, Acid Violet 43, Mordant Red 3; EXT violet No. 2;
and as examples of dyes of formula (V′), mention may be made of: Acid Black 48;
d) the nitro dyes of formula (VI) or (VI′):

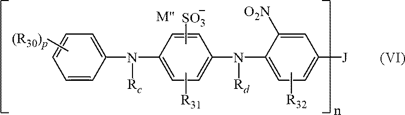

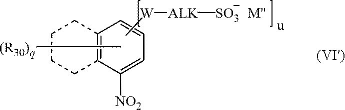

formulae (VI) and (VI′) wherein:
   ▪ R₃₀, R₃₁ and R₃₂, which may be identical or different, represent a hydrogen or halogen atom, or a group chosen from:
      (C₁-C₆)alkyl;
      (C₁-C₆)alkoxy optionally substituted with one or more hydroxyl group(s) or (C₁-C₆)alkylthio optionally substituted with one or more hydroxyl group(s);
      hydroxyl, mercapto;
      nitro, nitroso;
      polyhalo(C₁-C₆)alkyl;
      R°—C(X)—X′—, R°-X′-C(X)-, R°-X′-C(X)-X″- with R°, X, X′ and X″ as defined previously;
      — (O)₂S(O⁻)—, M⁺ with M⁺ as defined previously;
      — (O)CO⁻—, M⁺ with M⁺ as defined previously;
      (di)(C₁-C₆)(alkyl)amino;
      (di)(hydroxy(C₁-C₆)alkyl)amino;
      heterocycloalkyl such as piperidino, piperazino or morpholino;
      in particular, R₃₀, R₃₁ and R₃₂ represent a hydrogen atom;
   ▪ R_{c} and R_{d}, which may be identical or different, represent a hydrogen atom or a (C₁-C₆)alkyl group;
   ▪ W is as defined previously; W particularly represents an N(H)— group;
   ▪ ALK represents a linear or branched divalent C₁-C₆ alkylene group; in particular, ALK represents a —CH₂—CH₂— group;
   n is 1 or 2;
   ▪ p represents an integer ranging from 1 to 5;
   ▪ q represents an integer ranging from 1 to 4;
   u is 0 or 1;
   ▪ when n is 1, J represents a nitro or nitroso group; particularly nitro;
   ▪ when n is 2, J represents an oxygen or sulfur atom, or a divalent group —S(O)ₘ— with m representing an integer 1 or 2; preferentially, J represents an —SO₂— group;
   ▪ M″ represents a hydrogen atom or a cationic counterion; 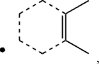
   which may be present or absent, represents a benzo group optionally substituted with one or more groups R₃₀ as defined previously;
      it being understood that the formulae (VI) and (VI′) comprise:
         at least one group (O)₂S(O⁻)—, M′⁺ with M′⁺ representing a cationic counterion; or
         at least one group (O)CO⁻—, M′⁺ with M′⁺ representing a cationic counterion; preferably at least one sodium sulfonate group.

As examples of dyes of formula (VI), mention may be made of: Acid Brown 13 and Acid Orange 3; as examples of dyes of formula (VI′), mention may be made of: Acid Yellow 1, sodium salt of 2,4-dinitro-1-naphthol-7-sulfonic acid, 2-piperidino-5-nitrobenzenesulfonic acid, 2-(4′-N,N-(2″-hydroxyethyl)amino-2′-nitro)anilineethanesulfonic acid, 4-β-hydroxyethylamino-3-nitrobenzenesulfonic acid; Ext. D&C Yellow 7;
e) the triarylmethane dyes of formula (VII):

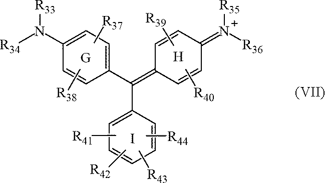

formula (VII) wherein:
   ▪ R₃₃, R₃₄, R₃₅ and R₃₆, which may be identical or different, represent a hydrogen atom or a group chosen from (C₁-C₆)alkyl, optionally substituted aryl and optionally substituted aryl(C₁-C₆)alkyl; particularly a (C₁-C₆)alkyl group and benzyl optionally substituted with an (O)ₘS(O⁻)—, M⁺ group with M⁺ and m as defined previously;
   ▪ R₃₇, R₃₈, R₃₉, R₄₀, R₄₁, R₄₂, R₄₃ and R₄₄, which may be identical or different, represent a hydrogen atom or a group chosen from:
      (C₁-C₆)alkyl;
      (C₁-C₆)alkoxy, (C₁-C₆)alkylthio;
      (di)(C₁-C₆)(alkyl)amino;
      hydroxyl, mercapto;
      nitro, nitroso;
      R°—C(X)—X′—, R°-X′-C(X)-, R°-X′-C(X)-X″- with R° representing a hydrogen atom, or an alkyl or aryl group; X, X′ and X″, which may be identical or different, representing an oxygen or sulfur atom, or NR with R representing a hydrogen atom or a (C₁-C₆)alkyl group;
      — (O)₂S(O⁻)—, M⁺ with M⁺ representing a hydrogen atom or a cationic counterion;
      — (O)CO⁻—, M⁺ with M⁺ as defined previously;
      or else two contiguous groups R₄₁ with R₄₂ or R₄₂ with R₄₃ or R₄₃ with R₄₄ together form a fused benzo group optionally substituted with one or more group(s) chosen from i) nitro; ii) nitroso; iii) (O)₂S(O⁻)—, M⁺; iv) hydroxyl; v) mercapto; vi) (di)(C₁-C₆)(alkyl)amino; vii) R°—C(X)—X′—; viii) R°-X′-C(X)-; ix) R°-X′-C(X)-X″-; with M⁺, R°, X, X′ and X″ as defined previously;
      particularly, R₃₇ to R₄₀ represent a hydrogen atom, and R₄₁ to R₄₄, which may be identical or different, represent a hydroxyl or (O)₂S(O⁻)—, M⁺ group with M⁺ as defined previously; and when R₄₃ with R₄₄ together form a benzo group, it is preferentially substituted with an (O)₂S(O⁻)— group; it being understood that at least one of the rings G, H or I comprises:
      at least one group (O)₂S(O⁻)—, M′⁺ with M′⁺ representing a cationic counterion; or
      at least one group (O)CO⁻—, M′⁺ with M′⁺ representing a cationic counterion; preferably at least one sodium sulfonate group.

As examples of dyes of formula (VII), mention may be made of: Acid Blue 1, Acid Blue 3, Acid Blue 7, Acid Blue 9, Acid Violet 49, Acid Green 3, Acid Green 5 and Acid Green 50.
f) the xanthene-based dyes of formula (VIII):

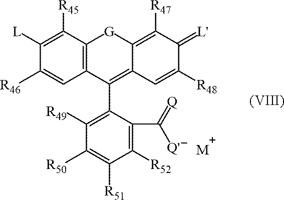

formula (VIII) wherein:
   ▪ R₄₅, R₄₆, R₄₇ and R₄₈, which may be identical or different, represent a hydrogen atom or a halogen atom;
   ▪ R₄₉, R₅₀, R₅₁ and R₅₂, which may be identical or different, represent a hydrogen or halogen atom, or a group chosen from:
      (C₁-C₆)alkyl;
      (C₁-C₆)alkoxy, (C₁-C₆)alkylthio;
      hydroxyl, mercapto;
      nitro, nitroso;
      — (O)₂S(O⁻)—, M⁺ with M⁺ representing a hydrogen atom or a cationic counterion;
      — (O)CO⁻—, M⁺ with M⁺ as defined previously;
      particularly, R₅₃, R₅₄, R₅₅ and R₄₈ represent a hydrogen or halogen atom;
   ▪ G represents an oxygen or sulfur atom or a group NRₑ with Rₑ as defined previously; particularly, G represents an oxygen atom;
   ▪ L represents an alkoxide O⁻, M⁺; a thioalkoxide S⁻, M⁺ or a group NR_{f}, with R_{f} representing a hydrogen atom or a (C₁-C₆)alkyl group, and M⁺ as defined previously; M⁺ is particularly sodium or potassium;
   ▪ L′ represents an oxygen or sulfur atom or an ammonium group: N⁺RᵣR_{g}, with R_{f} and R_{g}, which may be identical or different, representing a hydrogen atom, a (C₁-C₆)alkyl group or an optionally substituted aryl group; L′ represents particularly an oxygen atom or a phenylamino group optionally substituted with one or more alkyl or (O)ₘS(O⁻)—, M⁺ group(s) with m and M⁺ as defined previously;
   ▪ Q and Q′, which may be identical or different, represent an oxygen or sulfur atom; particularly Q and Q′ represent an oxygen atom;
   ▪ M⁺ is as defined previously.

As examples of dyes of formula (VIII), mention may be made of: Acid Yellow 73, Acid Red 51, Acid Red 52, Acid Red 87, Acid Red 92, Acid Red 95 and Acid Violet 9;
g) the indole-based dyes of formula (IX):

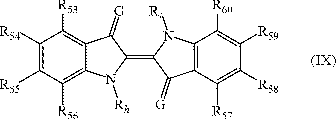

formula (IX) wherein:
   ▪ R₅₃, R₅₄, R₅₅, R₅₆, R₅₇, R₅₈, R₅₉ and R₆₀, which may be identical or different, represent a hydrogen atom or a group chosen from:
      (C₁-C₆)alkyl;
      (C₁-C₆)alkoxy, (C₁-C₆)alkylthio;
      hydroxyl, mercapto;
      nitro, nitroso;
      R°-C(X)-X′-, R°-X′-C(X)-, R°-X′-C(X)-X″with R° representing a hydrogen atom, or an alkyl or aryl group; X, X′ and X″, which may be identical or different, representing an oxygen or sulfur atom, or NR with R representing a hydrogen atom or a (C₁-C₆)alkyl group;
      — (O)₂S(O⁻)—, M⁺ with M⁺ representing a hydrogen atom or a cationic counterion;
      — (O)CO⁻—, M⁺ with M⁺ as defined previously;
   ▪ G represents an oxygen or sulfur atom or a group NRₑ with Rₑ as defined previously; particularly, G represents an oxygen atom;
   ▪ Rᵢ and Rₕ, which may be identical or different, represent a hydrogen atom or a (C₁-C₆)alkyl group;
   it being understood that formula (IX) comprises:
      at least one group (O)₂S(O⁻)—, M′⁺ with M′⁺ representing a cationic counterion; or
      at least one group (O)CO⁻—, M′⁺ with M′⁺ representing a cationic counterion; preferably at least one sodium sulfonate group.

As examples of dyes of formula (IX), mention may be made of: Acid Blue 74.
h) the quinoline-based dyes of formula (X):

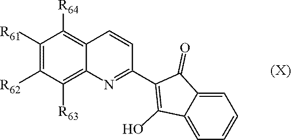

formula (X) wherein:
   ▪ R₆₁ represents a hydrogen or halogen atom or a (C₁-C₆)alkyl group;
   ▪ R₆₂, R₆₃ and R₆₄, which may be identical or different, represent a hydrogen atom or a group (O)₂S(O⁻)—, M⁺ with M⁺ representing a hydrogen atom or a cationic counterion; or else R₆₁ with R₆₂, or R₆₁ with R₆₄, together form a benzo group optionally substituted with one or more groups (O)₂S(O⁻)—, M⁺ with M⁺ representing a hydrogen atom or a cationic counterion;
   it being understood that formula (X) comprises at least one group (O)₂S(O⁻)—, M′⁺ with M′⁺ representing a cationic counterion, preferably at least one sodium sulfonate group. As examples of dyes of formula (X), mention may be made of: Acid Yellow 2, Acid Yellow 3 and Acid Yellow 5.

More particularly, the dyes of formulae (III) to (VIII) that are useful in the invention are chosen from: Acid Red 87 (VIII) (C.I. 45380); Sodium salt of 2,4-dinitro-1-naphthol-7-sulfonic acid (VI’) (C.I. 10316); Acid Orange 3 (VI) (C.I. 10383); Acid Yellow 9/Food Yellow 2 (III) (C.I. 13015); Direct Red 45/Food Red 13 (III) (C.I. 14780); Acid Black 52 (III) (C.I. 13711); Acid Yellow 36 (III) (C.I. 13065); Sodium salt of 1-hydroxy-2-(2′,4′-xylyl-5-sulfonatoazo)naphthalene-4-sulfonic acid/Food Red 1 (III) (C.I. 14700); Acid Red 14/Food Red 3/Mordant Blue 79 (III) (C.I. 14720); Sodium salt of 4-hydroxy-3-[(2-methoxy-5-nitrophenyl)diaza]-6-(phenylamino)naphthalene-2-sulfonic acid/Acid Brown 4 (III) (C. I. 14805); Acid Orange 7/Pigment Orange 17/Solvent Orange 49 (III) (C.I. 15510); Food Yellow 3/Pigment Yellow 104 (III) (C.I. 15985); Acid Red 27/Food Red 9 (III) (C.I. 16185); Acid Orange 10/Food Orange 4 (III) (C.I. 16230); Acid Red 44 (III) (C.I. 16250); Acid Red 33/Food Red 12 (III) (C.I. 17200); Acid Red 184 (III) (C.I. 15685); Acid Violet 3 (III) (C.I. 19125); Sodium salt of 1-hydroxy-2-(4′-acetamidophenylazo)-8-acetamidonaphthalene-3,6-disulfonic acid/Acid Violet 7/Food Red 11 (III) (C.I. 18055); Acid Red 135 (III) (C.I. 18130); Acid Yellow 27 (IV) (C.I. 19130); Acid Yellow 23/Food Yellow 4 (IV) (C.I. 19140); 4′-(sulfonato-2”,4″-dimethyl)-bis-(2,6-phenylazo)-1,3-dihydroxybenzene/Acid Orange 24 (III) (C.I. 20170); Sodium salt of 1-amino-2-(4′-nitrophenylazo)-7-phenylazo-8-hydroxynaphthalene-3,6-disulfonic acid/Acid Black 1 (III) (C.I. 20470); (4-((4-methylphenyl)sulfonyloxy)phenylazo)-2,2′-dimethyl-4-((2-hydroxy-5,8-disulfonato)naphthylazo)biphenyl/Acid Red 111 (III′) (C.I. 23266); Food Black 2 (III) (C.I. 27755); 1-(4′-sulfonatophenylazo)-4-((2″-hydroxy-3″-acetylamino-6”,8″-disulfonato)naphthylazo)-6-sulfonatonaphthalene (tetrasodium salt)/Food Black 1 (III) (C.I. 25440); Acid Blue 9 (VII) (C.I. 42090); Acid Violet 43 (V) (C.I. 60730); Acid Green 25 (V) (C.I. 61570); Sodium salt of 1-amino-4-cyclohexylamino-9,10-anthraquinone-2-sulfonic acid/Acid Blue 62 (V) (C.I. 62045); Acid Blue 78 (V) (C.I. 62105); Sodium salt of 4-hydroxy-3-((2-methoxyphenyl)azo)-1-naphthalenesulfonic acid/Acid Red 4 (III) (C.I. 14710); 2-Piperidino 5-nitrobenzenesulfonic acid (VI′); 2(4′-N,N(2″-hydroxyethyl)amino-2′-nitro)anilineethanesulfonic acid (VI′); 4-β-hydroxyethylamino-3-nitrobenzenesulfonic acid (VI’); Acid Violet 49 (VII) (C.I. 42640); Acid Blue 7 (VII) (C.I. 42080); Sodium salt of 1,2-dihydroxy-3-sulfoanthraquinone/Mordant Red 3 (V) (C.I. 58005); Sodium salt of 1-amino-9,10-dihydro-9,10-dioxo-4-(phenylamino) 2-anthracenesulfonic acid/Acid Blue 25 (V) (C.I. 62055); Sodium salt of 4-hydroxy-3-((2-methoxyphenyl)azo)-1-naphthalenesulfonic acid/Acid Red 4 (III) (C.I. 14710).

Most of these dyes are described in particular in the Color Index published by The Society of Dyers and Colorists, P.O. Box 244, Perkin House, 82 Grattan Road, Bradford, Yorkshire, BD1 2JBN England.

The anionic dyes that are most particularly preferred are the dyes designated in the Color Index under the code C.I. 58005 (monosodium salt of 1,2-dihydroxy-9,10-anthraquinone-3-sulfonic acid), C.I. 60730 (monosodium salt of 2-[(9,10-dihydro-4-hydroxy-9,10-dioxo-1-anthracenyl)amino]-5-methylbenzenesulfonic acid), C.I. 15510 (monosodium salt of 4-[(2-hydroxy-1-naphthalenyl)azo]benzenesulfonic acid), C.I. 15985 (disodium salt of 6-hydroxy-5-[(4-sulfophenyl)azo]-2-naphthalenesulfonic acid), C.I. 17200 (disodium salt of 5-amino-4-hydroxy-3-(phenylazo)-2,7-naphthalenedisulfonic acid), C.I. 20470 (disodium salt of 1-amino-2-(4′-nitrophenylazo)-7-phenylazo-8-hydroxy-3,6-naphthalenedisulfonic acid), C.I. 42090 (disodium salt of N-ethyl-N-[4-[[4-[ethyl(3-sulfophenyl)methyl]amino]phenyl](2-sulfophenyl)methylene]-2,5-cyclohexadien-1-ylidene]-3-sulfobenzenemethanaminium hydroxide, inner salt), C.I. 61570 (disodium salt of 2,2′-[(9,10-dihydro-9,10-dioxo-1,4-anthracenediyl)diimino]-bis-[5-methyl]benzenesulfonic acid).

Use may also be made of compounds corresponding to the mesomeric or tautomeric forms of structures (III) to (X).

### Natural Dyes

The direct dyes b) can be chosen from natural direct dyes.

Among the natural direct dyes that may be used according to the invention, mention may be made of lawsone, juglone, alizarin, purpurin, carminic acid, kermesic acid, purpurogallin, protocatechaldehyde, indigo, isatin, curcumin, spinulosin, apigenidin, orceins, brazilin, brazilein, hematein and hematoxylin. Extracts or decoctions containing these natural dyes and in particular henna-based poultices or extracts can also be used.

According to one preferred embodiment, the direct dyes b) are chosen from the triarylmethane direct dyes of formulae (IIa₁) and (IIa₂) below, and mixtures thereof:

| | | | |
|---|---|---|---|
| 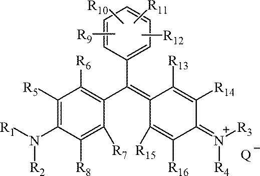 | (IIa₁ ) | 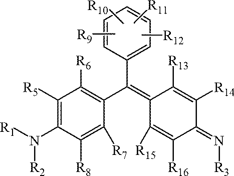 | (IIa₂) |

wherein:
▪ R₁, R₂, R₃ and R₄, which may be identical or different, represent a hydrogen atom or one of the following groups: (C₁-C₆)alkyl which is optionally substituted, preferably with a hydroxyl group; aryl such as phenyl, aryl(C₁-C₄)alkyl such as benzyl, heteroaryl, heteroaryl(C₁-C₄)alkyl, or else two groups R₁, and R₂, and/or R₃ and R₄, borne by the same nitrogen atom form, together with the nitrogen atom which bears them, an optionally substituted heterocycloalkyl group such as morpholino, piperazino or piperidino, preferably R₁, R₂, R₃ and R₄, which may be identical or different, represent a hydrogen atom or a (C₁-C₄)alkyl group;
▪ R₅, R₆, R₇, R₈, R₉, R₁₀, R₁₁, R₁₂, R₁₃, R₁₄, R₁₅, and R₁₆, which may be identical or different, represent a hydrogen or halogen atom, or a group chosen from i) hydroxyl, ii) thiol, iii) amino, iv) (di)(C₁-C₄)(alkyl)amino, v) (di)arylamino such as (di)phenylamino, vi) nitro, vii) acylamino (—NR—C(O)R′) wherein the R radical is a hydrogen atom, a C₁-C₄ alkyl radical optionally bearing at least one hydroxyl group and the R′ radical is a C₁-C₂ alkyl radical; viii) carbamoyl ((R)₂N—C(O)—) wherein the R radicals, which may be identical or different, represent a hydrogen atom or a C₁-C₄ alkyl radical optionally bearing at least one hydroxyl group; ix) carboxylic acid or ester, (—O—C(O)R′) or (—C(O)OR′), wherein the R′ radical is a hydrogen atom or C₁-C₄ alkyl optionally bearing at least one hydroxyl group and the R′ radical is a C₁-C₂ alkyl radical; x) alkyl which is optionally substituted, in particular with a hydroxyl group; xi) alkylsulfonylamino (R′SO₂—NR—) wherein the R radical represents a hydrogen atom or a C₁-C₄ alkyl radical optionally bearing at least one hydroxyl group and the R′ radical represents a C₁-C₄ alkyl radical, a phenyl radical; xii) aminosulfonyl ((R)₂N—SO₂—) wherein the R radicals, which may be identical or different, represent a hydrogen atom or a C₁-C₄ alkyl radical optionally bearing at least one hydroxyl group; xiii) (C₁-C₄)alkoxy; and xiv) (C₁-C₄)alkylthio;
▪ or else two radicals borne by two contiguous carbon atoms R₅ and R₆ and/or R₇ and R₈, and/or R₉ and R₁₀ and/or R₁₁ and R₁₂ and/or R₁₃ and R₁₄ and/or R₁₅ and R₁₆ form, together with the carbon atoms which bear them, an aryl or heteroaryl, preferably benzo, 6-membered fused ring, said ring possibly also being optionally substituted, preferably an unsubstituted benzo ring;
▪ Q⁻ represents an anionic counterion for achieving electrical neutrality, preferably chosen from halides such as chloride or bromide, and phosphate.

The direct dye(s) b) can preferably be chosen from Basic Violet 1, Basic Violet 2, Basic Violet 3, Basic Violet 4, Basic Violet 14, Basic Blue 1, Basic Blue 7, Basic Blue 26, Basic Green 1 and HC Blue 15, and mixtures thereof.

More preferentially, the direct dye b) is HC Blue 15.

The direct dye(s) b) may be present in the composition in a total content ranging from 0.001% to 10% by weight, preferably from 0.05% to 5% by weight, more preferentially from 0.1% to 3% by weight, relative to the total weight of the composition.

### Heterocyclic Salts of Formula (A)

The composition according to the invention comprises c) one or more heterocyclic salts of formula (A):

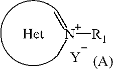

wherein:
▪ Het represents a cationic, aromatic, unsaturated heterocyclic group comprising:
   from 5 to 10 ring members, preferably 5 or 6 ring members; and
   in addition to the ammonium bearing R₁, one or two atoms chosen from nitrogen or oxygen atoms, preferably a nitrogen atom;
      said heterocyclic group being optionally substituted with one or more R′₁ or R₂ groups;
▪ R₁ and R′₁, which may be identical or different, represent a linear or branched, saturated or unsaturated C₁-C₁₂ hydrocarbon-based group optionally substituted notably with one or more groups chosen from hydroxyl, amino, (C₁-C₆)dialkylamino, (C₁-C₆)alkylamino, carboxyl, carboxylate, carbamide, (C₁-C₄)alkoxy, —SO₃H, sulfonate and phenyl;
▪ R₂ represents a hydroxyl radical, an amino radical, a linear or branched, saturated or unsaturated C₁-C₁₂ hydrocarbon-based group optionally substituted notably with one or more groups chosen from hydroxyl, amino, (C₁-C₆)dialkylamino, (C₁-C₆)alkylamino, carboxyl, carboxylate, carbamide, (C₁-C₄)alkoxy, —SO₃H, sulfonate and phenyl; and
= Y⁻ represents an anionic counterion;
   it being understood that:
      when one of the hydrocarbon-based groups of R₁, R′₁ or R₂ is substituted with a carboxylate or sulfonate group, then Y⁻ is absent to ensure the electrical neutrality of the salt of formula (A).

Preferably, the salt(s) of formula (A) are chosen from those of formula (I) below:

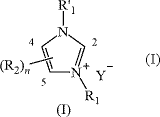

Formula (I) wherein:
▪ R₁ represents a linear or branched, saturated or unsaturated C₁-C₁₂ hydrocarbon-based group optionally substituted with one or more groups chosen from hydroxyl, amino, (C₁-C₆)dialkylamino, (C₁-C₆)alkylamino, carboxyl, carboxylate, carbamide, (C₁-C₄)alkoxy, —SO₃H, sulfonate and phenyl;
▪ R′₁ represents a hydrogen atom, a linear or branched, saturated or unsaturated C₁-C₁₂ hydrocarbon-based group optionally substituted with one or more groups chosen from hydroxyl, amino, (C₁-C₆)dialkylamino, (C₁-C₆)alkylamino, carboxyl, carboxylate, carbamide, (C₁-C₄)alkoxy, —SO₃H, sulfonate and phenyl; and
▪ R₂ represents a hydroxyl group, a linear or branched, saturated or unsaturated C₁-C₁₂ hydrocarbon-based group optionally substituted with one or more groups chosen from hydroxyl, (C₁-C₄)alkoxy, —SO₃H, sulfonate and benzene;
▪ n is 0, 1, 2 or 3, preferably n is 0 or 1;
▪ Y⁻ represents an anionic counterion;
   it being understood that:
      when one of the hydrocarbon-based groups of R₁, R′₁ or R₂ is substituted with a carboxylate or sulfonate group, then Y⁻ is absent to ensure the electrical neutrality of the salt of formula (A), and
      when n is 2 or 3, then the substituents are identical or different.

According to one particular embodiment, the salt(s) of formula (I) are such that R′₁ represents a hydrogen atom or a linear or branched, saturated or unsaturated C₁-C₈ hydrocarbon-based group optionally substituted with one or more groups chosen from hydroxyl, (C₁-C₂)alkoxy, —SO₃H, —SO₃⁻ and phenyl; preferably, R′₁ represents a hydrogen atom or a saturated, linear or branched, more preferentially linear, C₁-C₆ hydrocarbon-based group; even more preferentially, R′₁ represents a methyl group.

According to one particular embodiment, the salts of formula (I) are such that R₂ represents a linear or branched, saturated C₁-C₆ hydrocarbon-based group such as methyl, and preferably n is 1 and R₂ is in position 2.

According to one preferred embodiment of the invention, the salt(s) of formula (A) are chosen from the compounds 1 to 27 below, and mixtures thereof:

| 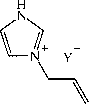 | 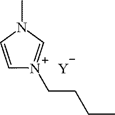 | 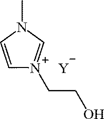 |
|---|---|---|
| 1 | 2 | 3 |
| 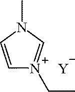 | 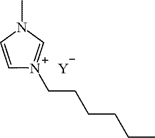 | 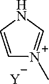 |
| 4 | 5 | 6 |
| 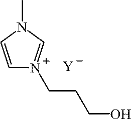 | 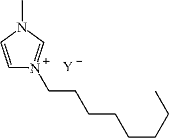 | 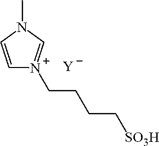 |
| 7 | 8 | 9 |
| 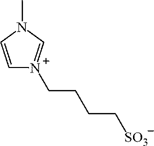 | | |
| 10 | | |
| 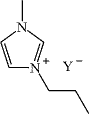 | 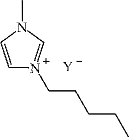 | 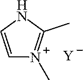 |
| 11 | 12 | 13 |
| 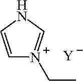 | 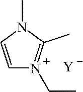 | 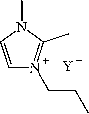 |
| 14 | 15 | 16 |
| 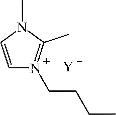 | 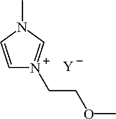 | 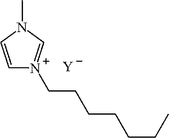 |
| 17 | 18 | 19 |
| 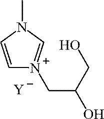 | 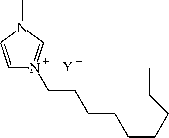 | 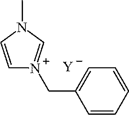 |
| 20 | 21 | 22 |
| 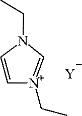 | 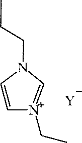 | 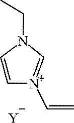 |
| 23 | 24 | 25 |
| 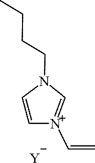 | 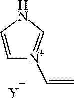 | |
| 26 | 27 | |

with Y⁻ representing an anionic counterion; preferably from the compounds 2, 3, 4, 5 and 25, and mixtures thereof, more preferentially from the compounds 2, 4, 5 and 25, and mixtures thereof, even more preferentially from the compounds 4 and mixtures thereof.

According to one preferred embodiment of the invention, the salt(s) of formula (A) are such that Y⁻ represents an anionic counterion chosen from i) halides such as chloride or bromide, ii) hydrogen sulfates, iii) (bis)(poly)halo(C₁-C₁₂)(alkyl)sulfonylimides such as bis(trifluoromethylsulfonyl)imide and bis(fluorosulfonyl)imide, iv) (C₁-C₁₂)alkyl sulfates, v) (poly)halophosphates such as hexafluorophosphate, vi) (C₁-C₁₂)(alkyl)phosphates such as phosphate, vii) (poly)haloborates such as tetrafluoroborate, viii) carbonate, ix) bicarbonate, x) (C₁-C₁₂)alkyl carbonates, xi) dicyanamide, xii) nitrate, xiii) thiocyanate, xiv) formate, xv) (C₁-C₁₂)alkyl carboxylates of which the (C₁-C₁₂)alkyl group can be substituted with one or more halogen atoms or groups chosen from hydroxyl, (C₁-C₆)(di)(alkyl)amino, phenyl, imidazole, (C₁-C₄)alkylcarbonyl, (C₁-C₄)alkylcarbonyloxy, (C₁-C₄)alkylcarbonylamino, guanidine, thiol, —SO₃H, (C₁-C₈)alkoxy such as trifluoroacetate; xvi) (C₁-C₂₀)alkene carboxylates of which the (C₁-C₂₀)alkene group can have one or more double bonds and can be substituted with one or more hydroxyl groups; xvii) (C₆-C₁₂)arylcarboxylates of which the aryl group can be substituted with one or more halogen atoms or groups chosen from hydroxyl, (C₁-C₆)(di)(alkyl)amino, phenyl, imidazole, (C₁-C₄)alkylcarbonyl, (C₁-C₄)alkylcarbonyloxy, (C₁-C₄)alkylcarbonylamino, guanidine, thiol, —SO₃H, (C₁-C₈)alkoxy; xviii) (C₁-C₁₂)alkylsulfonates of which the (C₁-C₁₂)alkyl group can be substituted with one or more halogen atoms or groups chosen from hydroxyl, (C₁-C₆)(di)(alkyl)amino, phenyl, imidazole, (C₁-C₄)alkylcarbonyle, (C₁-C₄)alkylcarbonyloxy, (C₁-C₄)alkylcarbonylamino, guanidine, thiol, —SO₃H, (C₁-C₈)alkoxy such as triflate, in particular (C₁-C₆)alkylsulfonates such as methylsulfonate or mesylate; and xix) (C₆-C₁₂)arylsulfonates of which the aryl group can be substituted with one or more halogen atoms or groups chosen from hydroxyl, (C₁-C₆)(di)(alkyl)amino, phenyl, imidazole, (C₁-C₄)alkylcarbonyl, (C₁-C₄)alkylcarbonyloxy, (C₁-C₄)alkylcarbonylamino, guanidine, thiol, —SO₃H, (C₁-C₈)alkoxy such as benzenesulfonate and toluenesulfonate or tosylate; preferably Y⁻ represents an anionic counterion chosen from:

| | | | |
|---|---|---|---|
| Cl⁻ | Br⁻ | CH₃SO₃⁻ | CH₃OSO₃⁻ |
| CH₃OCO₂- | N(CF₃SO₂)₂⁻ | N(SO₂F)₂⁻ | CF₃SO₃⁻ |
| PF₆⁻ | BF₄⁻ | N(CN)₂⁻ | (CH30)(H)PO₂⁻ |
| CH₃COO⁻ | HCO₃⁻ | NO₃⁻ | HCO₂⁻ |
| (CH₃O)₂PO₂⁻ | (CH₃CH2O)₂PO₂⁻ | HSO₄⁻ | (nC₄H₁₀O)₂PO₂⁻ |
| SCN⁻ | CF₃COO⁻ | H₂PO₄⁻ | CH₃CH₂OSO₃₋ |
| 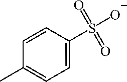 | 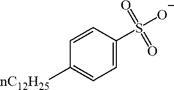 | 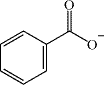 | 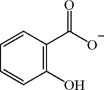 |
| 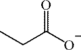 | 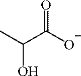 | 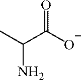 | 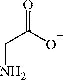 |
| 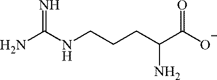 [SMILES: N=C(N)NCCCC(N)C(=O)[O-]] | | 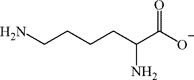 | |
| 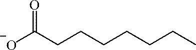 | | 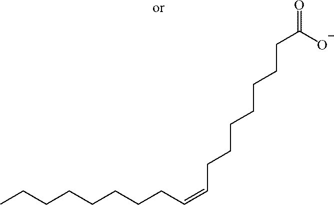 | |

more preferentially, Y⁻ represents an anionic counterion chosen from:

| | | | |
|---|---|---|---|
| CI⁻ | Br⁻ | CH₃OSO₃⁻ | 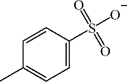 |
| N(CF₃SO₂)₂⁻ | | | |
| 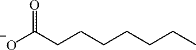 | | 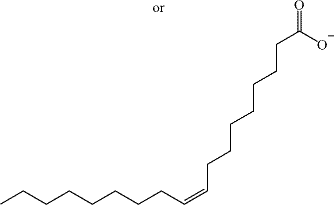 | |

even more preferentially Y⁻ represents an anionic counterion chosen from:

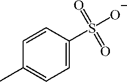

N(CF₃SO₂)₂⁻

| | |
|---|---|
| 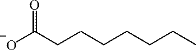 | 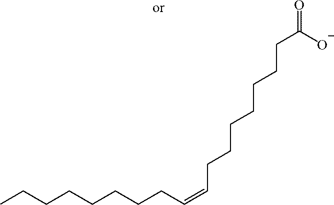 |

According to one particularly preferred embodiment, the salt(s) of formula (A) are chosen from the compounds 4a, 4b, 4c and 4d below, and mixtures thereof:

| | |
|---|---|
| 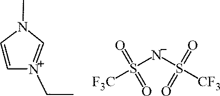 | 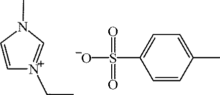 |
| 4a | 4b |
| 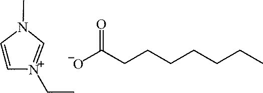 | 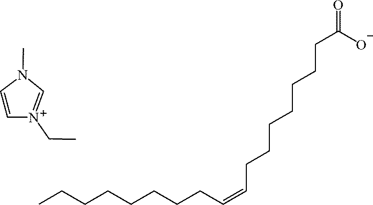 |
| 4c | 4d |

The salt(s) of formula (A) can be present in the composition in a total content ranging from 1% to 99.5% by weight, preferably from 3% to 90% by weight, more preferentially from 5% to 80% by weight, even more preferentially from 5% to 60% by weight, most preferentially from 5% to 50% by weight, and better still from 5% to 40% by weight relative to the total weight of the composition.

### Peroxygenated Salts D)

The composition according to the present invention preferably comprises d) one or more peroxygenated salts. The peroxygenated salts are preferably chosen from persulfates, perborates or percarbonates of alkali metals or alkaline-earth metals or of ammonium, and mixtures thereof, more preferentially from sodium, potassium or ammonium persulfates, and mixtures thereof.

The peroxygenated salt(s) d) are preferably present in the composition in a total content ranging from 0.1% to 50% by weight, preferably ranging from 1% to 25% by weight, relative to the total weight of the composition.

### Alkaline Agents E)

The composition according to the present invention preferably comprises e) one or more alkaline agents. The alkaline agents are preferably chosen from aqueous ammonia, alkanolamines such as monoethanolamine, urea, ammonium salts such as ammonium chloride, ammonium sulfate, ammonium phosphate or ammonium nitrate, and silicates, phosphates or carbonates of alkali metals or alkaline-earth metals, such as lithium, sodium, potassium, magnesium, calcium and barium, and mixtures thereof. Preferably, the alkaline agent(s) are chosen from aqueous ammonia, monoethanolamine, and silicates, such as sodium silicate, and mixtures thereof.

The alkaline agent(s) e) are preferably present in the composition in a total content ranging from 0.01% to 40% by weight, preferably from 0.1% to 30% by weight, relative to the total weight of the composition.

The composition according to the invention is a cosmetic composition, i.e. it is in a cosmetic medium.

The term “cosmetic medium” is intended to mean a medium that is suitable for dyeing keratin fibers, also known as a dye support, which is a cosmetic medium generally formed from water or from a mixture of water and one or more organic solvents or from a mixture of organic solvents.

The composition preferably comprises water in a content ranging from 5% to 99% by weight, more preferentially ranging from 5% to 95% by weight, relative to the total weight of the composition.

The term “organic solvent” is intended to mean an organic substance that is capable of dissolving another substance without chemically modifying it.

Mention may be made, as organic solvent, for example, of lower C₂-C₄ alkanols, such as ethanol and isopropanol; polyols and polyol ethers, such as 2-butoxyethanol, propylene glycol, propylene glycol monomethyl ether, diethylene glycol monoethyl ether and diethylene glycol monomethyl ether, and also aromatic alcohols, such as benzyl alcohol or phenoxyethanol, and mixtures thereof.

The organic solvents are present in proportions preferably ranging from 0.1% to 40% by weight, more preferentially from 1% to 30% by weight, even more preferentially from 1% to 25% by weight, relative to the total weight of the composition.

According to one preferred embodiment, the composition according to the invention comprises:
a) one or more chemical oxidizing agents chosen from hydrogen peroxide, hydrogen peroxide-generating agents other than peroxygenated salts, and mixtures thereof;
b) one or more direct dyes;
c) one or more heterocyclic salts of formula (A) as defined previously;
d) one or more peroxygenated salts;
e) one or more alkaline agents.

According to another preferred embodiment, the composition according to the invention comprises:
a) one or more chemical oxidizing agents chosen from hydrogen peroxide, hydrogen peroxide-generating agents other than peroxygenated salts, and mixtures thereof;
b) one or more direct dyes;
c) one or more heterocyclic salts of formula (A) as defined previously;
d) one or more peroxygenated salts;
e) one or more alkaline agents;
f) water or a mixture of water and one or more organic solvents chosen from lower C₂-C₄ alkanols, preferably ethanol.

### pH

The pH of the composition according to the invention is preferably from 3 to 12, more preferentially from 5 to 11, even more preferentially from 8 to 10.5.

The pH of this composition may be adjusted with acidifying or alkaline agents conventionally used in cosmetics.

Among the acidifying agents, examples that may be mentioned include organic or mineral acids.

The term “mineral acid” is intended to mean any acid derived from a mineral compound. Among the mineral acids, mention may be made of hydrochloric acid, orthophosphoric acid, sulfuric acid, sulfonic acids and nitric acid.

The term “organic acid” is intended to mean any acid derived from an organic compound. Among the organic acids, mention may be made of acetic acid, tartaric acid, citric acid, lactic acid and sulfonic acids.

Use may notably be made of mineral or organic acids such as hydrochloric acid, orthophosphoric acid, sulfuric acid, carboxylic acids, for instance acetic acid, tartaric acid, citric acid, lactic acid, and sulfonic acids.

Among the alkaline agents, examples that may be mentioned include aqueous ammonia, alkali metal carbonates, alkanolamines, such as monoethanolamine, diethanolamine and triethanolamine and derivatives thereof, sodium hydroxide, potassium hydroxide and the compounds of formula (B) below:

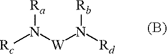

Formula (B) wherein W is a (C₁-C₆)alkylene group optionally substituted with one or more hydroxyl groups; Rₐ, R_{b}, R_{c} and R_{d}, which may be identical or different, represent a hydrogen atom or a (C₁-C₄)alkyl group optionally substituted with one or hydroxyl groups. Preferably, the pH modifiers may be chosen from alkaline agents, such as aqueous ammonia, monoethanolamine, diethanolamine, triethanolamine, 1,3-propanediamine or an alkaline hydroxide, such as 2-amino-2-methyl-1-propanol, or else acidifying agents, such as phosphoric acid or hydrochloric acid.

The composition according to the invention may be in liquid form, in the form of a serum, in thickened form, in particular a gel, a cream, a wax or a paste, or in foam form.

The composition according to the invention may also comprise one or more additional compounds chosen from nonionic, anionic, cationic or amphoteric surfactants, cationic, anionic, nonionic or zwitterionic, associative or nonassociative thickening polymers of natural or synthetic origin, silicones in the form of oil, gums or resins or non-silicone plant, mineral or synthetic oils, UV-screening agents, fillers, such as nacres and metal oxides such as titanium dioxides, clays, fragrances, peptizers, vitamins and preserving agents.

### Process for Simultaneous Bleaching and Dyeing of Keratin Fibers

According to a second aspect, a subject of the present invention is a process for simultaneous bleaching and dyeing of keratin fibers, comprising the application to the keratin fibers of a composition as defined previously.

The composition may be applied to wet or dry keratin fibers.

Preferably, the application to the keratin fibers of the composition according to the invention is carried out at ambient temperature, i.e. at a temperature of between 25° C. and 30° C.

The process can comprise a step consisting in leaving the composition on the keratin fibers for a time ranging from 5 min to 60 min.

According to one advantageous variant of the invention, after application of the composition or after the leave-on time, if present, has elapsed, the keratin fibers are rinsed, optionally shampooed, and then left to dry or dried, for example at a temperature of greater than or equal to 30° C.

According to one particular embodiment, this temperature is greater than 40° C. According to one particular embodiment, this temperature is greater than 45° C. and less than 220° C.

Preferably, if the keratin fibers are dried, they are dried, in addition to a supply of heat, with a flow of air.

During the drying, a mechanical action may be exerted on the fibers, such as combing, brushing or running the fingers through. This operation may similarly be carried out once the keratin fibers have dried, naturally or otherwise.

The drying step may be carried out with a drying device such as a hood, a hairdryer or a Climazon.

The drying step can be carried out with a hood or a hairdryer, the drying temperature ranging from 40° C. to 110° C., preferably from 50° C. to 90° C.

Once the drying is complete, final rinsing or shampooing may optionally be performed. The composition is applied to the wet or dry keratin fibers, preferably with a weight ratio of the amount of composition applied relative to the amount of hair ranging from 0.1 to 10, and more particularly ranging from 0.2 to 7.5.

### Kit

According to a third aspect, a subject of the present invention is a multi-compartment device or kit, comprising:
▪ a first compartment containing a composition comprising a) one or more chemical oxidizing agents chosen from hydrogen peroxide, hydrogen peroxide-generating agents other than peroxygenated salts, and mixtures thereof; and
▪ a second compartment containing a composition comprising:
   b) one or more direct dyes; and
   c) one or more salts of formula (A) as defined previously; or
▪ a first compartment containing a composition comprising a) one or more chemical oxidizing agents chosen from hydrogen peroxide, hydrogen peroxide-generating agents other than peroxygenated salts, and mixtures thereof; and
▪ a second compartment containing a composition comprising b) one or more direct dyes; and
▪ a third compartment containing a composition comprising c) one or more salts of formula (A) as defined previously.

In the embodiments according to which the composition of the invention comprises d) one or more peroxygenated salts and/or e) one or more alkaline agents, they may be present in the composition contained in the second or the third compartment.

The technical features defined previously regarding the various ingredients of the composition according to the present invention also apply to the ingredients of the kit.

### EXAMPLES

The examples that follow will allow the invention to be understood more clearly, without, however, being limiting in nature.

### Example 1

The following compositions were prepared and then applied according to the application protocol described below:

TABLE 1

| Ingredients | Composition 1 (Comparative) | Composition 2 (Comparative) | Composition 3 (Invention) |
|---|---|---|---|
| H₂O₂ at 12% in water (wt%) | 40 | 40 | 40 |
| Ammonium persulfate (wt%) | 7 | 7 | 7 |
| NH₄OH at 20% in water (wt%) | 20 | 20 | 20 |
| Ethanol (wt%) | 25 | 25 | 25 |
| 1-ethyl-3-methylimidazolium bis(trifluoromethanesulfonyl)imide (Compound 4a according to the invention) (wt%) | - | - | 5 |
| HC Blue 15 (wt%) | - | 0.1 | 0.1 |
| Water (wt%) | qs 100 | qs 100 | qs 100 |

### Application Protocol

In a trough, 10 g of composition 1, 2 or 3 are applied to a lock of 1 g of Caucasian hair with a tone depth of 4 (TD4) for 50 min at a temperature of 33° C. The lock of hair is then rinsed, shampooed and dried.

### Colorimetric Measurements

The color build-up (ΔE*) was evaluated in the CIE L* a* b* system, using a Minolta Spectrophotometer CM3610A colorimeter, (illuminant D65). In this L* a* b* system, L* represents the intensity of the color, a* indicates the shade of the color on the green/red color axis and b* indicates the shade of the color on the blue/yellow color axis. The lower the value of L*, the darker or more intense the color. The higher the value of a*, the redder the shade, and the higher the value of b*, the bluer the shade.

In the table below, the value of ΔE* is calculated from the L*a*b* values according to the following equation:

$$Δ E * = \sqrt{{\left(L * - {L}_{0} *\right)}^{2} + {\left(a * - {a}_{0} *\right)}^{2} + {\left(b * - {b}_{0} *\right)}^{2}}$$

In the equation, L*, a* and b* represent the values measured on the locks after treatment by means of the protocol above, and L₀*, a₀* and b₀* represent the values measured on control locks treated with composition 1.

The higher the value of ΔE*, the better the build-up of the dye.

### Results

The results of the measurements are indicated in the following table:

TABLE 2

| | L* | a* | b* | C* | h | ΔE* |
|---|---|---|---|---|---|---|
| Composition 1 (Comparative) | 42.07 | 11.69 | 26.17 | 28.67 | 65.93 | - |
| Composition 2 (Comparative) | 28.9 | 4.1 | 13.25 | 13.87 | 72.81 | 19.95 |
| Composition 3 (Invention) | 29.54 | -0.7 | 11.56 | 11.58 | 93.45 | 22.89 |

It is seen from the above results that the color build-up is significantly improved by the presence of a salt of formula (A) according to the present invention.

### Example 2

The following compositions were prepared and then applied according to the application protocol described below:

TABLE 3

| | | | |
|---|---|---|---|
| Ingredients | Composition 4 (Comparative) | Composition 5 (Comparative) | Composition 6 (Invention) |
| Oxidizing cream H₂O₂ 40v (wt%) | 50 | 50 | 50 |
| Sodium silicate (wt%) | 6.98 | 6.98 | 6.98 |
| Potassium persulfate (wt%) | 6.35 | 6.35 | 6.35 |
| 1-ethyl-3-methylimidazolium tosylate (Compound 4b according to the invention) (wt%) | - | - | 10 |
| HC Blue 15 (wt%) | - | 0.5 | 0.5 |
| water (wt%) | qs 100 | qs 100 | qs 100 |

### Application Protocol

In a trough, 10 g of composition 4, 5 or 6 are applied to a lock of 1 g of TD4 Caucasian hair for 50 min at a temperature of 33° C. The lock of hair is then rinsed, shampooed and dried.

### Colorimetric Measurements

The color build-up (ΔE*) was evaluated in the CIE L* a* b* system, using a Minolta Spectrophotometer CM3610A colorimeter, (illuminant D65). In this L* a* b* system, L* represents the intensity of the color, a* indicates the shade of the color on the green/red color axis and b* indicates the shade of the color on the blue/yellow color axis. The lower the value of L*, the darker or more intense the color. The higher the value of a*, the redder the shade, and the higher the value of b*, the bluer the shade.

In the table below, the value of ΔE* is calculated from the L*a*b* values according to the following equation:

$$Δ E * = \sqrt{{\left(L * - {L}_{0} *\right)}^{2} + {\left(a * - {a}_{0} *\right)}^{2} + {\left(b * - {b}_{0} *\right)}^{2}}$$

In the equation, L*, a* and b* represent the values measured on the locks after treatment by means of the protocol above, and L₀*, a₀* and b₀* represent the values measured on control locks treated with composition 4.

The higher the value of ΔE*, the better the build-up of the dye.

### Results

The results of the measurements are indicated in the following table:

TABLE 4

| | L* | a* | b* | C* | h | ΔE* |
|---|---|---|---|---|---|---|
| Composition 4 (Comparative) | 34.2 | 10.02 | 19.63 | 22.04 | 62.96 | - |
| Composition 5 (Comparative) | 35.48 | 0.48 | 15.77 | 15.78 | 88.27 | 10.37 |
| Composition 6 (Invention) | 25.88 | 0.33 | 9.11 | 9.11 | 87.94 | 16.55 |

It is seen from the above results that the color build-up is significantly improved by the presence of a salt of formula (A) according to the present invention.

### Example 3

The following compositions were prepared and then applied according to the application protocol described below:

TABLE 5

| | | | |
|---|---|---|---|
| Ingredients | Composition 7 (Comparative) | Composition 8 (Comparative) | Composition 9 (Invention) |
| H₂O₂ at 12% in water (wt%) | 40 | 40 | 40 |
| Ammonium persulfate (wt%) | 7 | 7 | 7 |
| NH₄OH at 20% in water (wt%) | 20 | 20 | 20 |
| Ethanol (wt%) | 25 | 25 | 25 |
| 1-ethyl-3-methylimidazolium octanoate (Compound 4c according to the invention) (wt%) | - | - | 5 |
| HC BLUE 15 (wt%) | - | 0.1 | 0.1 |
| Water (wt%) | Qs 100 | Qs 100 | Qs 100 |

### Application Protocol

In a trough, 10 g of composition 7, 8 or 9 are applied to a lock of 1 g of 90% white natural Caucasian hair for 50 min at a temperature of 33° C. The lock of hair is then rinsed, shampooed and dried.

### Colorimetric Measurements

The color build-up (ΔE*) was evaluated in the CIE L* a* b* system, using a Minolta Spectrophotometer CM3610A colorimeter, (illuminant D65). In this L* a* b* system, L* represents the intensity of the color, a* indicates the shade of the color on the green/red color axis and b* indicates the shade of the color on the blue/yellow color axis. The lower the value of L*, the darker or more intense the color. The higher the value of a*, the redder the shade, and the higher the value of b*, the bluer the shade.

In the table below, the value of ΔE* is calculated from the L*a*b* values according to the following equation:

$$Δ E * = \sqrt{{\left(L * - {L}_{0} *\right)}^{2} + {\left(a * - {a}_{0} *\right)}^{2} + {\left(b * - {b}_{0} *\right)}^{2}}$$

In the equation, L*, a* and b* represent the values measured on the locks after treatment by means of the protocol above, and L₀*, a₀* and b₀* represent the values measured on control locks treated with composition 7.

The higher the value of ΔE*, the better the build-up of the dye.

### Results

The results of the measurements are indicated in the following table:

TABLE 6

| | L* | a* | b* | C* | h | ΔE* |
|---|---|---|---|---|---|---|
| Composition 7 (Comparative) | 66.71 | 2.38 | 15.51 | 15.7 | 81.26 | - |
| Composition 8 (Comparative) | 57.06 | -7.65 | 5.8 | 9.6 | 142.81 | 16.97 |
| Composition 9 (Invention) | 59.98 | -13.45 | -1.46 | 13.53 | 186.2 | 24.17 |

It is seen from the above results that the color build-up is significantly improved by the presence of a salt of formula (A) according to the present invention.

### Example 4

The following compositions were prepared and then applied according to the application protocol described below:

TABLE 7

| | | | |
|---|---|---|---|
| Ingredients | Composition 10 (Comparative) | Composition 11 (Comparative) | Composition 12 (Invention) |
| H₂O₂ at 12% in water (wt%) | 40 | 40 | 40 |
| Ammonium persulfate (wt%) | 7 | 7 | 7 |
| NH₄OH at 20% in water (wt%) | 20 | 20 | 20 |
| Ethanol (wt%) | 25 | 25 | 25 |
| 1-ethyl-3-methylimidazolium octadec-9-enoate (wt%) (Compound 4d according to the invention) (wt%) | - | - | 5 |
| HC BLUE 15 (wt%) | - | 0.1 | 0.1 |
| Water (wt%) | Qsp 100 | Qs 100 | Qs 100 |

### Application Protocol

In a trough, 10 g of composition 10, 11 or 12 are applied to a lock of 1 g of 90% white natural Caucasian hair for 50 min at a temperature of 33° C. The lock of hair is then rinsed, shampooed and dried.

### Colorimetric Measurements

The color build-up (ΔE*) was evaluated in the CIE L* a* b* system, using a Minolta Spectrophotometer CM3610A colorimeter, (illuminant D65). In this L* a* b* system, L* represents the intensity of the color, a* indicates the shade of the color on the green/red color axis and b* indicates the shade of the color on the blue/yellow color axis. The lower the value of L*, the darker or more intense the color. The higher the value of a*, the redder the shade, and the higher the value of b*, the bluer the shade.

In the table below, the value of ΔE* is calculated from the L*a*b* values according to the following equation:

$$Δ E * = \sqrt{{\left(L * - {L}_{0} *\right)}^{2} + {\left(a * - {a}_{0} *\right)}^{2} + {\left(b * - {b}_{0} *\right)}^{2}}$$

In the equation, L*, a* and b* represent the values measured on the locks after treatment by means of the protocol above, and L₀*, a₀* and b₀* represent the values measured on control locks treated with composition 10.

The higher the value of ΔE*, the better the build-up of the dye.

### Results

The results of the measurements are indicated in the following table:

TABLE 8

| | L* | a* | b* | C* | h | ΔE* |
|---|---|---|---|---|---|---|
| Composition 10 (Comparative) | 66.71 | 2.38 | 15.51 | 15.7 | 81.26 | - |
| Composition 11 (Comparative) | 57.06 | -7.65 | 5.8 | 9.6 | 142.81 | 16.97 |
| Composition 12 (Invention) | 58.1 | -10.05 | 1.92 | 10.23 | 169.2 | 20.33 |

It is seen from the above results that the color build-up is significantly improved by the presence of a salt of formula (A) according to the present invention.

1-18. (canceled)

19. A composition comprising:
a) one or more chemical oxidizing agents chosen from hydrogen peroxide, hydrogen peroxide-generating agents other than peroxygenated salts, or mixtures of two or more thereof;
b) one or more direct dyes; and
c) one or more heterocyclic salts of formula (A): 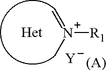
wherein:
■ Het is a cationic, aromatic, unsaturated heterocyclic group comprising:
from 5 to 10 ring members; and
one or two atoms chosen from nitrogen or oxygen, in addition to the ammonium bearing R₁;
wherein Het is optionally substituted with one or more R′₁ or R₂ groups;
■ R₁ and R′₁, which may be identical or different, are chosen from C₁-C₁₂ hydrocarbon-based groups, optionally substituted with one or more groups chosen from hydroxyl, amino, (C₁-C₆)dialkylamino, (C₁-C₆)alkylamino, carboxyl, carboxylate, carbamide, (C₁-C₄)alkoxy, —SO₃H, sulfonate, or phenyl;
■ R₂ is chosen from a hydroxyl radical, an amino radical, or a C₁-C₁₂ hydrocarbon-based group optionally substituted with one or more groups chosen from hydroxyl, amino, (C₁-C₆)dialkylamino, (C₁-C₆)alkylamino, carboxyl, carboxylate, carbamide, (C₁-C₄)alkoxy, —SO₃H, sulfonate, or phenyl; and
■ Y⁻ represents an anionic counterion;
with the proviso that when one of the hydrocarbon-based groups of R₁, R′₁, or R₂ is substituted with a carboxylate or sulfonate group, then Y⁻ is absent.

20. The composition according to claim 19, wherein the chemical oxidizing agent comprises hydrogen peroxide.

21. The composition according to claim 19, wherein the total amount of chemical oxidizing agents a) ranges from 0.1% to 40% by weight, relative to the total weight of the composition.

22. The composition according to claim 19, further comprising d) one or more peroxygenated salts.

23. The composition according to claim 22, wherein the total amount of peroxygenated salts d) ranges from 0.1% to 50% by weight, relative to the total weight of the composition.

24. The composition according to claim 19, further comprising e) one or more alkaline agents.

25. The composition according to claim 24, wherein the total amount of alkaline agents e) ranges from 0.01% to 40% by weight, relative to the total weight of the composition.

26. The composition according to claim 19, comprising one or more salts of formula (A) chosen from compounds of formula (I) below: 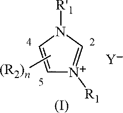
wherein:
■ R₁ is chosen from C₁-C₁₂ hydrocarbon-based groups optionally substituted with one or more groups chosen from hydroxyl, amino, (C₁-C₆)dialkylamino, (C₁-C₆)alkylamino, carboxyl, carboxylate, carbamide, (C₁-C₄)alkoxy, —SO₃H, sulfonate, or phenyl;
■ R′₁ is chosen from a hydrogen atom or a linear or branched, saturated or unsaturated C₁-C₁₂ hydrocarbon-based group optionally substituted with one or more groups chosen from hydroxyl, amino, (C₁-C₆)dialkylamino, (C₁-C₆)alkylamino, carboxyl, carboxylate, carbamide, (C₁-C₄)alkoxy, —SO₃H, sulfonate, or phenyl;
■ R₂ is chosen from a hydroxyl group or a linear or branched, saturated or unsaturated C₁-C₁₂ hydrocarbon-based group optionally substituted with one or more groups chosen from hydroxyl, (C₁-C₄)alkoxy, —SO₃H, sulfonate, or benzene;
■ n is 0, 1, 2, or 3; and
■ Y⁻ is an anionic counterion;
with the provisos that:
when one of the hydrocarbon-based groups of R₁, R′₁, or R₂ is substituted with a carboxylate or sulfonate group, then Y⁻ is absent, and
when n is 2 or 3, then R₁, R′₁, or R₂ may be identical or different.

27. The composition according to claim 26, wherein R′₁ is chosen from a hydrogen atom or a linear or branched, saturated or unsaturated C₁-C₈ hydrocarbon-based group optionally substituted with one or more groups chosen from hydroxyl, (C₁-C₂)alkoxy, —SO₃H, —SO₃⁻, or phenyl.

28. The composition according to claim 26, wherein R₂ is a linear or branched, saturated C₁-C₆ hydrocarbon-based group.

29. The composition according to claim 26, comprising one or more salts of formula (A) chosen from compounds 1 to 27:

| 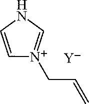 | 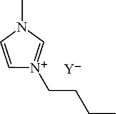 | 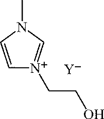 |
|---|---|---|
| 1 | 2 | 3 |
| 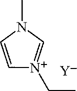 | 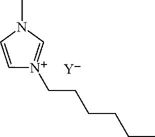 | 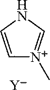 |
| 4 | 5 | 6 |
| 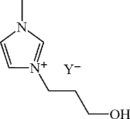 | 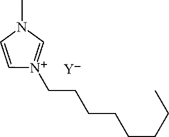 | 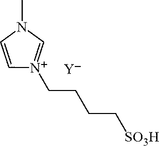 |
| 7 | 8 | 9 |
| 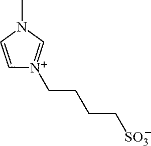 | | |
| 10 | | |
| 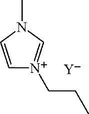 | 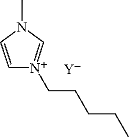 | 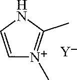 |
| 11 | 12 | 13 |
| 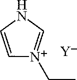 | 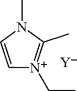 | 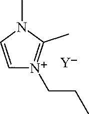 |
| 14 | 15 | 16 |
| 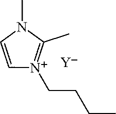 | 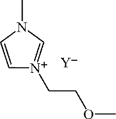 | 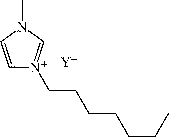 |
| 17 | 18 | 19 |
| 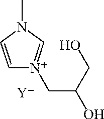 | 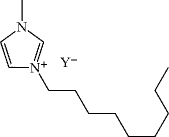 | 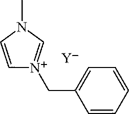 |
| 20 | 21 | 22 |
| 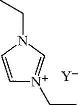 | 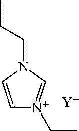 | 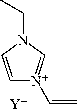 |
| 23 | 24 | 25 |
| 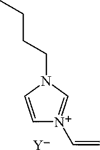 | 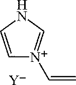 | |
| 26 | 27 | |

wherein Y
⁻ is an anionic counterion.

30. The composition according to claim 29, comprising one or more salts of formula (A) chosen from compounds 4a, 4b, 4c, or 4d:

| | | |
|---|---|---|
| 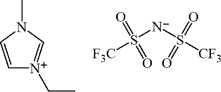 | | 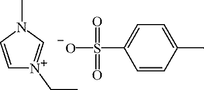 |
| 4a | | 4b |
| 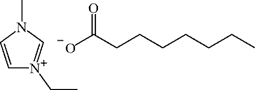 | 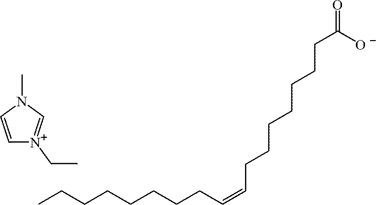 | |
| 4c | 4d. | |

31. The composition according to claim 26, wherein Y⁻ represents an anionic counterion chosen from:
i) halides,
ii) hydrogen sulfates,
iii) (bis)(poly)halo(C₁-C₁₂)(alkyl)sulfonylimides,
iv) (C₁-C₁₂)alkyl sulfates,
v) (poly)halophosphates,
vi) (C₁-C₁₂)(alkyl)phosphates,
vii) (poly)haloborates,
viii) carbonate, ix) bicarbonate,
x) (C₁-C₁₂)alkyl carbonates,
xi) dicyanamide,
xii) nitrate,
xiii) thiocyanate,
xiv) formate,
xv) (C₁-C₁₂)alkyl carboxylates, wherein the (C₁-C₁₂)alkyl group is optionally substituted with one or more halogen atoms or groups chosen from hydroxyl, (C₁-C₆)(di)(alkyl)amino, phenyl, imidazole, (C₁-C₄)alkylcarbonyl, (C₁-C₄)alkylcarbonyloxy, (C₁-C₄)alkylcarbonylamino, guanidine, thiol, —SO₃H, or (C₁-C₈)alkoxy;
xvi) (C₁-C₂₀)alkene carboxylates, wherein the (C₁-C₂₀)alkene group optionally has one or more double bonds and is optionally substituted with one or more hydroxyl groups;
xvii) (C₆-C₁₂)arylcarboxylates, wherein the aryl group is optionally substituted with one or more halogen atoms or groups chosen from hydroxyl, (C₁-C₆)(di)(alkyl)amino, phenyl, imidazole, (C₁-C₄)alkylcarbonyl, (C₁-C₄)alkylcarbonyloxy, (C₁-C₄)alkylcarbonylamino, guanidine, thiol, —SO₃H, or (C₁-C₈)alkoxy;
xviii) (C₁-C₁₂)alkylsulfonates, wherein the (C₁-C₁₂)alkyl group is optionally substituted with one or more halogen atoms or groups chosen from hydroxyl, (C₁-C₆)(di)(alkyl)amino, phenyl, imidazole, (C₁-C₄)alkylcarbonyle, (C₁-C₄)alkylcarbonyloxy, (C₁-C₄)alkylcarbonylamino, guanidine, thiol, —SO₃H, or (C₁-C₈)alkoxy; or
xix) (C₆-C₁₂)arylsulfonates of which the aryl group is optionally substituted with one or more halogen atoms or groups chosen from hydroxyl, (C₁-C₆)(di)(alkyl)amino, phenyl, imidazole, (C₁-C₄)alkylcarbonyl, (C₁-C₄)alkylcarbonyloxy, (C₁-C₄)alkylcarbonylamino, guanidine, thiol, —SO₃H, (C₁-C₈)alkoxy.

32. The composition of claim 31, wherein Y⁻ represents an anionic counterion chosen from:

| | | | |
|---|---|---|---|
| Cl⁻ | Br | CH₃SO₃⁻ | CH₃OSO₃⁻ |
| CH₃OCO₂- | N(CF₃SO₂)₂⁻ | N(SO₂F)₂⁻ | CF₃SO₃⁻ |
| PF₆⁻ | BF₄⁻ | N(CN)₂⁻ | (CH₃O)(H)PO₂⁻ |
| CH₃COO⁻ | HCO₃⁻ | NO₃⁻ | HCO₂⁻ |
| (CH₃O)₂PO₂⁻ | (CH₃CH₂O)₂PO₂⁻ | HSO₄⁻ | (nC₄H₁₀O)₂PO₂⁻ |
| SCN- | CF₃COO⁻ | H₂PO₄⁻ | CH₃CH₂OSO₃⁻ |
| 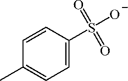 | 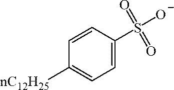 | 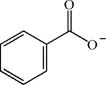 | 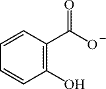 |
| 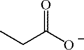 | 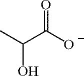 | 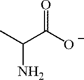 | 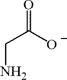 |
| 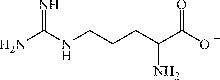 [SMILES: N=C(N)NCCCC(N)C(=O)[O-]] | | 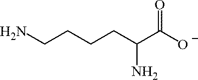 | |
| 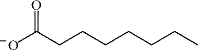 | | 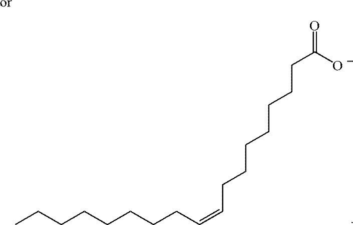 | |

.

33. The composition according to claim 32, wherein the total amount of salts of formula (A) ranges from 5% to 60% by weight, relative to the total weight of the composition.

34. The composition according to claim 19, comprising one or more direct dyes b) chosen from triarylmethane direct dyes of formula (IIa₁) or formula (IIa₂):

| | |
|---|---|
| 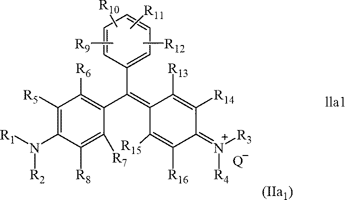 | 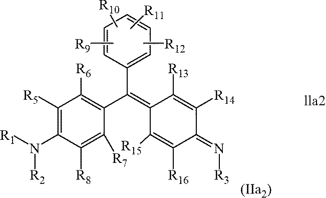 |

wherein:
■ R₁, R₂, R₃, and R₄, which may be identical or different:
are chosen from a hydrogen atom, (C₁-C₆)alkyl which is optionally substituted with a hydroxyl group, aryl, aryl(C₁-C₄)alkyl, heteroaryl, or heteroaryl(C₁-C₄)alkyl, or
two groups R₁ and R₂, and/or R₃ and R₄, together with the nitrogen atom which bears them, form an optionally substituted heterocycloalkyl group;
■ R₅, R₆, R₇, R₈, R₉, R₁₀, R₁₁, R₁₂, R₁₃, R₁₄, R₁₅, and R₁₆, which may be identical or different:
are chosen from a hydrogen atom, a halogen atom, or a group chosen from i) hydroxyl, ii) thiol, iii) amino, iv) (di)(C₁-C₄)(alkyl)amino, v) (di)arylamino, vi) nitro, vii) acylamino (—NR—C(O)R′) wherein the R radical is a hydrogen atom, viii) carbamoyl ((R)₂N—C(O)—) wherein the R radicals, which may be identical or different, are chosen from a hydrogen atom or a C₁-C₄ alkyl radical optionally bearing at least one hydroxyl group, ix) carboxylic acid or ester, (—O—C(O)R′) or (—C(O)OR′), wherein the R′ radical is a hydrogen atom or C₁-C₄ alkyl optionally bearing at least one hydroxyl group and the R′ radical is a C₁-C₂ alkyl radical, x) alkyl which is optionally substituted with a hydroxyl group, xi) alkylsulfonylamino (R′SO₂—NR—) wherein the R radical is chosen from a hydrogen atom or a C₁-C₄ alkyl radical optionally bearing at least one hydroxyl group and the R′ radical is chosen from a C₁-C₄ alkyl radical or a phenyl radical, xii) aminosulfonyl ((R)₂N—SO₂—) wherein the R radicals, which may be identical or different, are chosen from a hydrogen atom or a C₁-C₄ alkyl radical optionally bearing at least one hydroxyl group, xiii) (C₁-C₄)alkoxy, or xiv) (C₁-C₄)alkylthio, or
two radicals borne by two contiguous carbon atoms R5 and R6 and/or R7 and R8, and/or R9 and R10 and/or R11 and R12 and/or R13 and R14 and/or R15 and R16 form, together with the carbon atoms which bear them, form an aryl or heteroaryl; and
■ Q⁻ is an anionic counterion in order to achieve electrical neutrality.

35. The composition according to claim 19, wherein the total amount of direct dyes b) ranges from 0.05% to 5% by weight, relative to the total weight of the composition.

36. The composition according to claim 19, wherein the pH of the composition ranges from 5 to 11.

37. A method for simultaneous bleaching and dyeing of keratin fibers, the method comprising applying to the keratin fibers a composition comprising:
a) one or more chemical oxidizing agents chosen from hydrogen peroxide, hydrogen peroxide-generating agents other than peroxygenated salts, or mixtures of two or more thereof;
b) one or more direct dyes; and
c) one or more heterocyclic salts of formula (A): 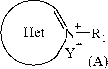
wherein:
■ Het is a cationic, aromatic, unsaturated heterocyclic group comprising:
from 5 to 10 ring members; and
one or two atoms chosen from nitrogen or oxygen, in addition to the ammonium bearing R₁;
wherein Het is optionally substituted with one or more R′₁ or R₂ groups;
■ R₁ and R′₁, which may be identical or different, are chosen from C₁-C₁₂ hydrocarbon-based groups, optionally substituted with one or more groups chosen from hydroxyl, amino, (C₁-C₆)dialkylamino, (C₁-C₆)alkylamino, carboxyl, carboxylate, carbamide, (C₁-C₄)alkoxy, —SO₃H, sulfonate, or phenyl;
■ R₂ is chosen from a hydroxyl radical, an amino radical, or a C₁-C₁₂ hydrocarbon-based group optionally substituted with one or more groups chosen from hydroxyl, amino, (C₁-C₆)dialkylamino, (C₁-C₆)alkylamino, carboxyl, carboxylate, carbamide, (C₁-C₄)alkoxy, —SO₃H, sulfonate, or phenyl; and
■ Y⁻ represents an anionic counterion;
with the proviso that when one of the hydrocarbon-based groups of R₁, R′₁ or R₂ is substituted with a carboxylate or sulfonate group, then Y⁻ is absent.

38. A multi-compartment kit, comprising:
(i) a first compartment comprising a) one or more chemical oxidizing agents chosen from hydrogen peroxide, hydrogen peroxide-generating agents other than peroxygenated salts, or mixtures of two or more thereof; and
(ii) a second compartment comprising:
b) one or more direct dyes; and
c) optionally, one or more heterocyclic salts of formula (A): 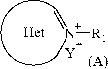
wherein:
■ Het is a cationic, aromatic, unsaturated heterocyclic group comprising:
from 5 to 10 ring members; and
one or two atoms chosen from nitrogen or oxygen, in addition to the ammonium bearing R₁;
wherein Het is optionally substituted with one or more R′₁ or R₂ groups;
■ R₁ and R′₁, which may be identical or different, are chosen from C₁-C₁₂ hydrocarbon-based groups, optionally substituted with one or more groups chosen from hydroxyl, amino, (C₁-C₆)dialkylamino, (C₁-C₆)alkylamino, carboxyl, carboxylate, carbamide, (C₁-C₄)alkoxy, —SO₃H, sulfonate, or phenyl;
■ R₂ is chosen from a hydroxyl radical, an amino radical, or a C₁-C₁₂ hydrocarbon-based group optionally substituted with one or more groups chosen from hydroxyl, amino, (C₁-C₆)dialkylamino, (C₁-C₆)alkylamino, carboxyl, carboxylate, carbamide, (C₁-C₄)alkoxy, —SO₃H, sulfonate, or phenyl; and
■ Y⁻ represents an anionic counterion;
with the provisos that:
when one of the hydrocarbon-based groups of R₁, R′₁, or R₂ is substituted with a carboxylate or sulfonate group, then Y⁻ is absent; and
when the c) one or more heterocyclic salts of formula (A) are not present in the second compartment (ii), the multi-compartment kit further comprises (iii) a third compartment comprising the c) one or more heterocyclic salts of formula (A).
